(19) United States
(12) Patent Application Publication
(10) Pub. No.: US 2007/0281935 A1
(43) Pub. Date: Dec. 6, 2007

(54) Use

(75) Inventor: Marcus Eugene Kehrli JR., Ames, IA (US)

Correspondence Address: PHARMACIA & UPJOHN, 7000 Portage Road, KZO-300-104, KALAMAZOO, MI, 49001, US

(73) Assignee: PFIZER INC., New York, NY (US)

(21) Appl. No.: 11/569,513

(22) PCT Filed: May 13, 2005
(86) PCT No.: PCT/IB05/01438

§ 371 Date: Aug. 23, 2007

Related U.S. Application Data
(60) Provisional application No. 60/574,171, filed on May 25, 2004.

(51) Int. Cl. A61K 31/445; A61K 31/45; A61K 31/453; A61K 31/4535; A61K 31/4545; A61P 43/00

(52) U.S. Cl. 514/235.5; 514/253.11; 514/317; 514/318; 514/319; 514/326; 514/327

(57) ABSTRACT

The use of a compound of formula I: 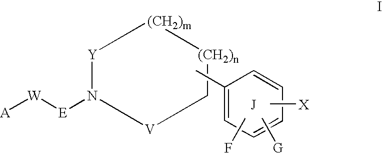 an isomer thereof, a prodrug of said compound or isomer, or a pharmaceutically acceptable salt of said compound, isomer or prodrug, in the manufacture of a medicament for the palliative, prophylactic or curative treatment of negative energy balance in ruminants. The use of a compound of formula 1, in the manufacture of a medicament for the palliative, prophylactic or curative treatment of ruminant disease associated with negative energy balance in ruminants, wherein, preferably, the ruminant disease associated with negative energy balance in ruminants is selected from fatty liver syndrome, dystocia, immune dysfunction, impaired immune function, toxification, primary ketosis, secondary ketosis, downer cow syndrome, indigestion, inappetence, retained placenta, displaced abomasum, mastitis, (endo-)-metritis, infertility, low fertility, and lameness.

### FIELD OF THE INVENTION

The invention described herein relates to the novel use of peroxisome proliferator-activated receptor (PPAR) agonists, in particular PPAR alpha agonists, for the treatment of negative energy balance in ruminants, and more particularly for the treatment of disease associated with negative energy balance (NEB) in ruminants.

### BACKGROUND TO THE INVENTION

The ruminant transition period is defined as the period spanning late gestation to early lactation. This is sometimes defined as from 3 weeks before to three weeks after parturition, but has been expanded to 30 days prepartum to 70 days postpartum (J N Spain and W A Scheer, Tri-State Dairy Nutrition Conference, 2001, 13).

Energy balance is defined as energy intake minus energy output and an animal is described as being in negative energy balance if energy intake is insufficient to meet the demands on maintenance and production (eg milk). A cow in NEB has to find the energy to meet the deficit from its body reserves. Thus cows in NEB tend to lose body condition and liveweight, with cows that are more energy deficient tending to lose condition and weight at a faster rate.

It is important that the mineral and energy balance and overall health of the cow is managed well in the transition period, since this interval is critically important to the subsequent health, production, and profitability in dairy cows.

Ruminants rely almost exclusively on gluconeogenesis in the liver to meet their glucose requirements, since unlike in monogastric mammals, little glucose is absorbed directly from the digestive tract. Feed intake is diminished around calving and insuffient propionate, the major glucogenic precursor formed in the rumen, is available. Catabolism of amino acids from the diet or from skeletal muscle also contributes significantly to glucose synthesis.

Long chain fatty acids (or non esterified fatty acids, NEFAs) are also mobilised from body fat. NEFAs, already elevated from around 7 days prepartum, are a significant source of energy to the cow during the early postpartum period, and the greater the energy deficit the higher the concentration of NEFA in the blood. Some workers suggest that in early lactation (Bell and references therein-see above) mammary uptake of NEFAs accounts for some milk fat synthesis. The circulating NEFAs are taken up by the liver and are oxidised to carbon dioxide or ketone bodies, including 3-hydroxybutyrate, by mitochondria, or reconverted via esterification into triglycerides and stored. In non-ruminant mammals it is thought that entry of NEFAs into the mitochondria is controlled by the enzyme carnitine palmitoyltransferase (CPT-1) however, some studies have shown that in ruminants there is little change in activity of CPT-1 during the transition period (Drackley—see above) Furthermore, the capacity of the liver for synthesising very low density lipoproteins to export triglycerides from the liver is limited.

Significantly, if NEFA uptake by the bovine liver becomes excessive, accumulation of ketone bodies can lead to ketosis, and excessive storage of triglycerides may lead to fatty liver. Fatty liver can lead to prolonged recovery for other disorders, increased incidence of health problems, and development of “downer cows” that die.

Thus, fatty liver is a metabolic disease of ruminants, particularly high producing dairy cows, in the transition period that negatively impacts disease resistance (abomasal displacement, lameness), immune function (mastitits, metritis), reproductive performance (oestrus, calving interval, foetal viability, ovarian cysts, metritis, retained placenta), and milk production (peak milk yield, 305 day milk yield). Fatty liver has largely developed by the day after parturition and precedes an induced (secondary) ketosis. It usually results from increased esterification of NEFA absorbed from blood coupled with the low ability of ruminant liver to secrete triglycerides as very low-density lipoproteins.

By improving energy balance, or by treating the negative energy balance, the negative extent of the sequelae will be reduced.

In humans, chronic administration of stimulators of PPAR alpha (peroxisome proliferator activated receptor alpha) activity can provide therapeutic benefits for the treatment of dyslipidemia, coronary artery disease, and certain hereditary enzyme deficiencies (P. T. Ines, P. Gervois, B. Staels, Current Opinion Lipidology, 1999, 10, 2, 151). However, many biological, metabolic and physiological pathways differ between monogastric mammals and ruminants. One typical and important example in the context of this application is the energy metabolism, since microbes in the rumen almost exclusively digest carbohydrates in the food. The main sources for carbohydrates in cows are therefore volatile fatty acids that are re-synthesised to glucose in the liver.

The PPAR alpha gene has also been implicated in a number of metabolic processes by regulating genes involved in gluconeogenesis, ketogenesis, fatty acid uptake and oxidation in mammals, (M. C. Sugden, K. Bulmer, G. F. Gibbons, B. L. Knight, M. J. Holness, Biochem J., 2002, 364, 361).

Most recently Drackley has hypothesised that high fat diets prepartum may have increased PPAR alpha expression, resulting in increased hepatic oxidation and decreased esterification of fatty acids in transition cow liver tissue. However, the interplay of biological processes is complicated as described, and knowledge of the important genes, enzymes and endogenous substrates required to optimise the energy balance in transition cows is limited. Furthermore, it is not known how modification of PPAR expression will effect milk production or quality, lipolysis or gluconeogenesis, since NEFA's are critical substrates for both milk and glucose biosynthesis.

There is a general need for a safe, effective treatment of negative energy balance in ruminants. In particular, there is a need for a treatment for ruminants such as sheep and cattle, more particularly for periparturient sheep and cattle, especially for periparturient dairy cows.

More particularly, there is a need for a safe, effective treatment of ruminant disease associated with negative energy balance in ruminants, which include primary and secondary ketosis, downer cow syndrome, indigestion, inappetence, retained placenta, displaced abomasum, impaired immune function, mastitis, (endo-)-metritis, infertility, low fertility, lameness, subacute rumen acidosis and inadequate nutrient intake associated with stress e.g. heat, poor housing, overcrowding, shipping, dominance or illness.

The treatment is preferably administered easily orally or parenterally, preferably does not present residues in meat and/or milk, and preferably does not require a withholding period. It is also preferably non-toxic to feed and animal handlers.

We have discovered a novel use of a compound of formula I, for the palliative, prophylactic or curative treatment of negative energy balance in ruminants. In particular, we have discovered a novel use of a compound of formula I, for the palliative, prophylactic or curative treatment of ruminant disease associated with negative energy balance in ruminants.

One aspect of the invention is the use of a compound of formula I, an isomer thereof, a prodrug of said compound or isomer, or a pharmaceutically acceptable salt of said compound, isomer or prodrug, in the manufacture of a medicament for the palliative, prophylactic or curative treatment of negative energy balance in ruminants.

Another aspect of the invention is a method of palliative, prophylactic or curative treatment of negative energy balance in ruminants, which comprises administration to a ruminant of an effective amount of a compound of formula I, an isomer thereof, a prodrug of said compound or isomer, or a pharmaceutically acceptable salt of said compound, isomer or prodrug.

Further aspects of the invention are as defined in the description and claims.

U.S. Provisional Patent Application No. (US) 60/574171, unpublished at the priority date of the present invention, which was published with International Patent Application Publication Number (WO) 04/048334, describes PPAR activators which are described to be useful in various disorders including cardiovascular and metabolic disorders.

U.S. Provisional Patent Application No. (US) 60/574136, which shares the priority date of the present invention, discloses the use of PPAR agonists in raising glucose serum levels in ruminants.

### DESCRIPTION OF THE DRAWINGS

FIG. 1 shows bovine liver triglyceride content after parturition, and after administration of a compound of formula I.

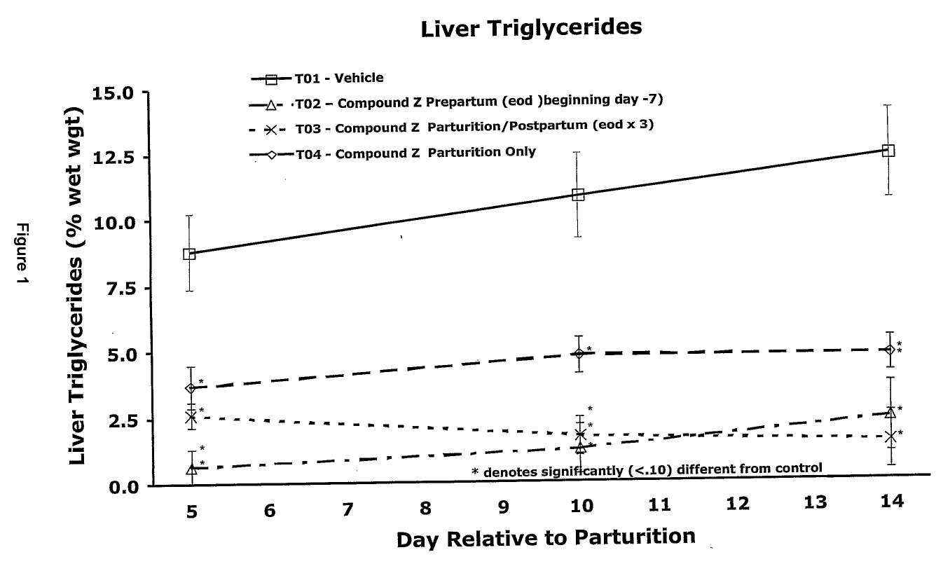

FIG. 2 shows bovine serum NEFA levels after parturition, and after administration of a compound of formula I.

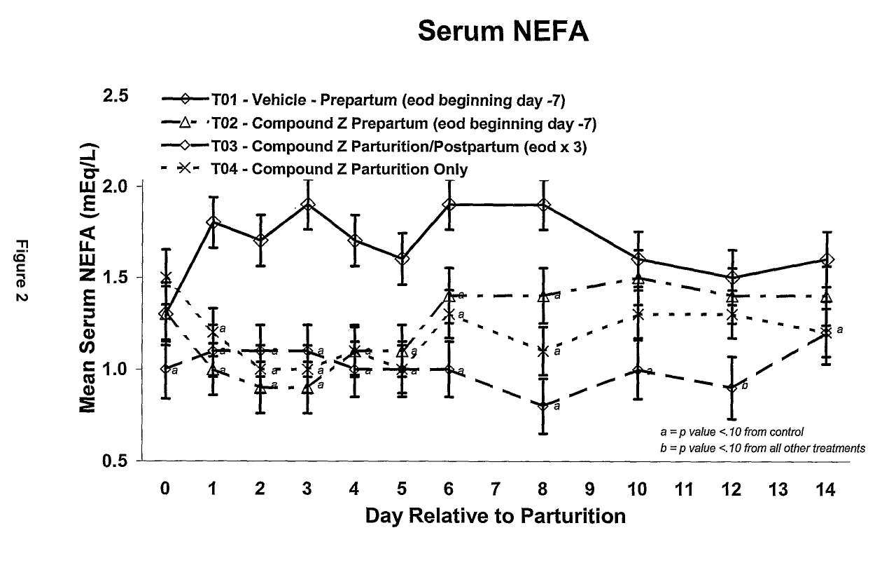

FIG. 3 describes the average daily milk yield compared to placebo in one hundred twenty four pregnant, non-lactating cows treated by a PPAR agonist.

FIG. 4 describes the weekly mean protein yield compared to placebo in one hundred twenty four pregnant, non-lactating cows treated by a PPAR agonist.

### SUMMARY OF THE INVENTION

As a first aspect, the present invention provides the use of a compound of formula I, as described in US 60/574171 and WO 04/048334:

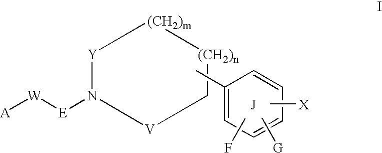

isomers thereof, prodrugs of said compounds or isomers, or pharmaceutically acceptable salts of said compounds, isomers or prodrugs;

wherein m and n are each independently one or two;

V and Y are each independently a) methylene, or b) carbonyl;

F and G are each independently a) hydrogen, b) halo, c) (C₁-C₄)alkyl optionally substituted with one to nine fluoro, d) (C₃-C₆)cycloalkyl, e) hydroxy, f) (C₁-C₄)alkoxy or g) (C₁-C₄)alkylthio;

X is a) —Z or b) —B—C(R¹R²)—Z;

B is a) oxy, b) thio, c) sulfinyl, d) sulfonyl, e) methylene, or f) —N(H)—;

Z is a) —C(O)OH, b) —C(O)O-(C₁-C₄)alkyl, c) —C(O)O-(C₀-C₄)alkyl-aryl, d) —C(O)—NH₂, e) hydroxyaminocarbonyl, f) tetrazolyl, g) tetrazolylaminocarbonyl, h) 4,5-dihydro-5-oxo-1,2,4-oxadiazol-3-yl, i) 3-oxoisoxazolidin-4-yl-aminocarbonyl, j) —C(O)N(H)SO₂R⁴, or k) —NHSO₂R⁴ wherein R⁴ is a) (C₁-C₆)alkyl, b) amino or c) mono-N- or di-N,N-(C₁-C₆)alkylamino, wherein the (C₁-C₆)alkyl substituents in R⁴ are optionally independently substituted with one to nine fluoro;

R¹ is a) H, b) (C₁-C₄)alkyl, or c) (C₃-C₆)cycloalkyl;

R² is a) H, b) (C₃-C₆)cycloalkyl or c) a fully or partially saturated or fully unsaturated one to four membered straight or branched carbon chain; wherein the carbon(s) in the carbon chain may optionally be replaced with one or two heteroatoms selected independently from oxygen and sulfur; and wherein the sulfur is optionally mono- or di-substituted with oxo;

wherein the carbon(s) in the carbon chain in R² is optionally independently substituted as follows: a) the carbon(s) is optionally mono-, di- or tri-substituted independently with halo, b) the carbon(s) is optionally mono-substituted with hydroxy or (C₁-C₄)alkoxy, and c) the carbon(s) is optionally mono-substituted with oxo; and

wherein the carbon(s) in the carbon chain in R² is optionally mono-substituted with Q;

wherein Q is a partially or fully saturated or fully unsaturated three to eight membered ring optionally having one to four heteroatoms selected independently from oxygen, sulfur and nitrogen, or is a bicyclic ring consisting of two fused partially or fully saturated or fully unsaturated three to six membered rings, taken independently; wherein the bicyclic ring optionally has one to four heteroatoms selected independently from oxygen, sulfur and nitrogen;

wherein the Q ring is optionally mono-, di- or tri-substituted independently with a) halo, b) (C₂-C₆)alkenyl, c) (C₁-C₆) alkyl, d) hydroxy, e) (C₁-C₆)alkoxy, f) (C₁-C₄)alkylthio, g) amino, h) nitro, i) cyano, j) oxo, k) carboxy, l) (C₁-C₆)alkyloxycarbonyl, or m) mono-N- or di-N,N-(C₁-C₆)alkylamino; wherein the (C₁-C₆)alkyl and (C₁-C₆)alkoxy substituents on the Q ring is optionally mono-, di- or tri-substituted independently with a) halo, b) hydroxy, c) (C₁-C₆)alkoxy, d) (C₁-C₄)alkylthio, e) amino, f) nitro, g) cyano, h) oxo, i) carboxy, j) (C₁-C₆)alkyloxycarbonyl, or k) mono-N- or di-N,N-(C₁-C₆)alkylamino; wherein the (C₁-C₆)alkyl substituent is on the Q ring is also optionally substituted with one to nine fluoro;

or wherein R¹ and R² are linked together to form a three to six membered fully saturated carbocyclic ring, optionally having one heteroatom selected from oxygen, sulfur and nitrogen to form a heterocyclic ring;

E is a) carbonyl, b) sulfonyl, or c) methylene;

W is a) a bond, b) carbonyl, c) —N(H)—, d) —N((C₁-C₄)alkyl)-, e) (C₂-C₈)alkenyl, f) oxy, g) —(C₁-C₄)alkyl-O-, h) —NH-(C₁-C₄)alkyl-, or i) -(C₁-C₆)alkyl-; wherein the (C₁-C₆)alkyl and the (C₂-C₈)alkenyl groups in W may optionally be mono- or di-substituted independently with a) oxo, b) halo, c) (C₁-C₆)alkoxycarbonyl, d) (C₁-C₆)alkyl, e) (C₂-C₆)alkenyl, f) (C₃-C₇)cycloalkyl, g) hydroxy, h) (C₁-C₆)alkoxy, i) (C₁-C₄)alkylthio, j) amino, k) cyano, l) nitro, m) mono-N- or di-N,N-(C₁-C₆)alkylamino, or n) —NH-(C₁-C)alkylamino;

or wherein W is CR⁷R⁸ wherein R⁷ and R⁸ are linked together to form a three to six membered fully saturated carbocyclic ring;

A is a) mono-N- or di-N,N-(C₁-C₆)alkylamino, b) (C₂-C₆)alkanoylamino, c) (C₁-C₆)alkoxy, d) a partially or fully saturated or fully unsaturated three to eight membered ring optionally having one to four heteroatoms selected independently from oxygen, sulfur and nitrogen, or e) a bicyclic ring consisting of two fused partially or fully saturated or fully unsaturated three to six membered rings, taken independently; wherein the bicyclic ring optionally has one to four heteroatoms selected independently from oxygen, sulfur and nitrogen; and

wherein the A ring is optionally mono-, di- or tri-substituted independently with a) oxo, b) carboxy, c) halo, d) (C₁-C₆)alkoxycarbonyl, e) (C₁-C₆)alkyl, f) (C₂-C₆)alkenyl, g) (C₃-C₇)cycloalkyl, h) (C₃-C₇)cycloalkyl(C₁-C₆)alkyl, i) hydroxy, j) (C₁-C₆)alkoxy, k) (C₁-C₄)alkylthio, l) (C₁-C₄)alkylsulfonyl, m) amino, n) cyano, o) nitro, or p) mono-N- or di-N,N-(C₁-C₆)alkylamino; wherein the (C₁-C₆)alkyl and (C₁-C₆)alkoxy substituents on the A ring are also optionally mono-, di- or tri-substituted independently with a) halo, b) hydroxy, c) (C₁-C₄)alkyl optionally substituted with one to nine fluoro, d) (C₃-C₆)cycloalkyl, e) (C₁-C₆)alkoxy, f) amino, or g) mono-N- or di-N,N-(C₁-C₆)alkylamino;

or wherein the A ring is optionally mono-substituted with a partially or fully saturated or fully unsaturated three to eight membered ring, optionally having one to four heteroatoms selected independently from oxygen, sulfur and nitrogen; also wherein this three to eight membered ring is optionally mono-, di- or tri-substituted independently with a) halo, b) hydroxy, c) (C₁-C₄)alkyl optionally substituted with one to nine fluoro, d) (C₃-C₇)cycloalkyl, e) (C₁-C₆)alkoxy optionally substituted with one to nine fluoro, f) amino, g) mono-N- or di-N,N-(C₁-C₆)alkylamino, or h) (C₁-C₄)alkylthio;

provided that:

1) when V and Y are each methylene and m and n are each one forming a six-membered piperidinyl ring, this ring is substituted by the phenyl ring (designated as J) at other than the 4-position;

2) when E is carbonyl, W is a bond and X is —B—C(R¹R²)—Z wherein R¹ and R² are each hydrogen, B is —O— or —N(H)—, and Z is —C(O)OH or —C(O)O-(C₁-C₄)alkyl, then one of F or G must be a) -(C₁-C₄)alkyl, b) (C₃-C₆)cycloalkyl, c) (C₁-C₄)alkoxy or d) (C₁-C₄)alkylthio,

in the manufacture of a medicament for the palliative, prophylactic or curative treatment of negative energy balance in ruminants.

More particularly, the present invention provides the use of a compound of formula I with the further proviso that:

3) when E is carbonyl, W is a bond, X is —Z, and Z is —C(O)OH, —C(O)O-(C₁-C₄)alkyl, —C(O)NH₂, then one of F or G must be a) -(C₁-C₄)alkyl, b) (C₃-C₆)cycloalkyl, c) (C₁-C₄)alkoxy or d) (C₁-C₄)alkylthio.

More particularly, the present invention provides the use of a compound of formula I wherein V and Y are each methylene; or wherein one of V and Y is carbonyl and the other is methylene.

More particularly, the present invention provides the use of a compound of formula I wherein

E is carbonyl;

W is a) a bond, b) oxy, c) —N(H)—, d) —N(H)-(C₁-C₄)alkyl-, e) -(C₁-C₄)alkyl-, f) -(C₁-C₄)alkyl-O- or g) —CR⁷R⁸— wherein R⁷ and R⁸ are linked together to form a three-membered fully saturated carbocyclic ring; and

A is a partially or fully saturated or fully unsaturated three to eight membered ring optionally having one to four heteroatoms selected independently from oxygen, sulfur and nitrogen;

wherein the A ring is optionally mono-, di- or tri-substituted independently with a) oxo, b) carboxy, c) halo, d) (C₁-C₆)alkoxycarbonyl, e) (C₁-C₆)alkyl, f) (C₂-C₆)alkenyl, g) (C₃-C₇)cycloalkyl, h) (C₃-C₇)cycloalkyl(C₁-C₆)alkyl, i) hydroxy, j) (C₁-C₆)alkoxy, k) (C₁-C₄)alkylthio, l) (C₁-C₄)alkylsulfonyl, m) amino, n) cyano, o) nitro, or p) mono-N- or di-N,N-(C₁-C₆)alkylamino; wherein the (C₁-C₆)alkyl and (C₁-C₆)alkoxy substituents on the A ring are also optionally mono-, di- or tri-substituted independently with a) halo, b) hydroxy, c) (C₁-C₄)alkyl optionally substituted with one to nine fluoro, d) (C₃-C₆)cycloalkyl, e) (C₁-C₆)alkoxy, f) amino, or g) mono-N- or di-N,N-(C₁-C₆)alkylamino;

or wherein the A ring is optionally mono-substituted with a partially or fully saturated or fully unsaturated three to eight membered ring, optionally having one to four heteroatoms selected independently from oxygen, sulfur and nitrogen; also wherein this three to eight membered ring is optionally mono-, di- or tri-substituted independently with a) halo, b) hydroxy, c) (C₁-C₆)alkyl optionally substituted with one to nine fluoro, d) (C₃-C₇)cycloalkyl, e) (C₁-C₆)alkoxy optionally substituted with one to nine fluoro, f) amino, g) mono-N- or di-N,N-(C₁-C₆)alkylamino, or h) (C₁-C₄)alkylthio;

More particularly, the present invention provides the use of a compound of formula I wherein

A is a) phenyl optionally independently substituted with one or two 1) -(C₁-C₆)alkyl, 2) —CF₃, 3) —OCF₃ 4) -(C₁-C₆)alkoxy, 5) (C₃-C₇)cycloalkyl, 6) halo, 7) -(C₁-C₄)alkylthio or 8) hydroxy; or b) thiazolyl optionally independently substituted with 1) one or two methyl or 2) phenyl optionally independently substituted with one or two a) -(C₁-C₆)alkyl, b) —OCF₃, c) —OCF₃, d) -(C₁-C₆)alkoxy, e) (C₃-C₇)cycloalkyl, f) halo, g) -(C₁-C₄)alkylthio or h) hydroxy.

More particularly, the present invention provides the use of a compound of formula I wherein

F and G are each independently a) hydrogen, b) halo, c) (C₁-C₄)alkyl or d) (C₁-C₄)alkoxy;

X is a) —Z or b) —B—C(R¹R²)—Z;

B is a) oxy, b) thio or c) —N(H)—;

Z is a) —C(O)OH, b) —C(O)O-(C₁-C₄)alkyl, c) —C(O)NH₂ or d) tetrazolyl;

R¹ is a) hydrogen or b) methyl; and

R² is a) hydrogen or b) a fully or partially saturated or fully unsaturated one to four membered straight or branched carbon chain; wherein the carbon(s) in the carbon chain may optionally be replaced with one or two heteroatoms selected independently from oxygen and sulfur;

wherein the carbon(s) in the carbon chain in R² is optionally mono-substituted with Q;

wherein Q is a partially or fully saturated or fully unsaturated three to eight membered ring optionally having one to four heteroatoms selected independently from oxygen, sulfur and nitrogen.

More particularly, the present invention provides the use of a compound of formula I wherein

R¹ is a) hydrogen or b) methyl; and

R² is a) hydrogen, b) methyl or c) —O—CH₂-phenyl.

More particularly, the present invention provides compounds wherein

m is one, n is one and V and Y are each methylene to form a piperdinyl ring;

X is —B—C(R¹R²)—Z;

B is oxy; and

the phenyl ring (designated as J) is attached at the 3-position of the piperidinyl ring.

In particular, the present invention provides the use of a compound of formula I-A wherein

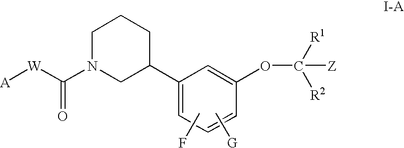

wherein R¹ and R² are each independently a) hydrogen or b) methyl;

F and G are each independently a) hydrogen or b) methyl; and

Z is —C(O)OH.

In particular, the present invention provides the use of such compounds of formula I-A wherein

W is a) oxy, b) —N(H)—, c) —N(H)-(C₁-C₄)alkyl-, d) -(C₁-C₄)alkyl- or e) -(C₁-C₄)alkyl-O-; and

A is phenyl optionally substituted with a) -(C₁-C₄)alkyl, b) —CF₃, c) —OCF₃ d) -(C₁-C₄)alkoxy, e) cyclopropyl, f) halo, g) -(C₁-C₄)alkylthio or h) hydroxy.

In particular, the present invention also provides the use of such compounds of formula I-A wherein

W is a bond; and

A is thiazolyl optionally substituted with a) one or two -methyl, or b) -phenyl optionally substituted with 1) -(C₁-C₄)alkyl, 2) —CF₃, 3) —OCF₃ 4) -(C₁-C₄)alkoxy, 5) cyclopropyl, 6) halo or 7) -(C₁-C₄)alkylthio.

More particularly, the present invention provides the use of a compound of formula I wherein

m is one, n is one and V and Y are each methylene to form a piperidinyl ring;

X is —Z;

and

the phenyl ring (designated as J) is attached at the 3-position of the piperidinyl ring.
In particular, the present invention provides the use of a compound of formula I-B

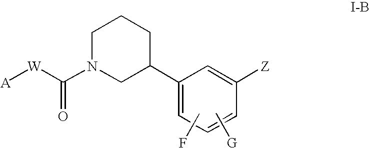

wherein F and G are each a) hydrogen, b) methyl, c) fluoro or d) methoxy; and

Z is a) —C(O)OH, b) —C(O)O-(C₁-C₄)alkyl or c) —C(O)NH₂.

More particularly, the present invention provides compounds of formula I-B wherein

W is a) -(C₁-C₄)alkyl- or b) -(C₁-C₄)alkyl-O-; and

A is phenyl optionally substituted with a) -(C₁-C₄)alkyl, b) —CF₃, c) —OCF₃, d) -(C₁-C₄)alkoxy, e) cyclopropyl, f) halo or g) hydroxy.

More particularly, the present invention provides the use of a compound of formula I-B wherein

W is a bond; and

A is thiazolyl optionally substituted with a) one or two -methyl or b) -phenyl optionally substituted with 1) -(C₁-C₄)alkyl, 2) —CF₃, 3) —OCF₃ 4) -(C₁-C₄)alkoxy, 5) cyclopropyl or 6) halo.

In particular, the present invention provides the use of compounds of formula I-C 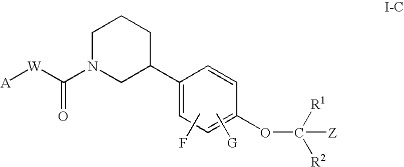
wherein R¹ and R² are each independently a) hydrogen or b) methyl;

F and G are each independently a) hydrogen or b) methyl; and

Z is —C(O)OH.

More particularly, the present invention provides the use of a compound of formula I-C wherein

W is a) oxy, b) —N(H)—, c) —N(H)-(C₁-C₄)alkyl, d) -(C₁-C₄)alkyl- or e) -(C₁-C₄)alkyl-O-; and

A is phenyl optionally substituted with a) -(C₁-C₄)alkyl, b) —CF₃, c) —OCF₃ d) -(C₁-C₄)alkoxy, e) cyclopropyl, f) halo, g) -(C₁-C₄)alkylthio or h) hydroxy.

More particularly, the present invention also provides the use of compound of formula I-C wherein

W is a bond; and

A is thiazolyl optionally substituted with a) one or two -methyl or b) -phenyl optionally substituted with 1) -(C₁-C₄)alkyl, 2) —CF₃, 3) —OCF₃ 4) -(C₁-C₄)alkoxy, 5) cyclopropyl, 6) halo or 7) -(C₁-C₄)alkylthio.

In particular, the present invention provides the use of a compound of formula I-D

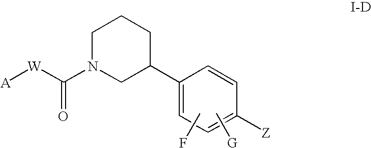

wherein F and G are each independently a) hydrogen, b) methyl, c) fluoro or d) methoxy; and

Z is a) —C(O)OH, b) —C(O)O-(C₁-C₄)alkyl or c) —C(O)NH₂.

More particularly, the present invention provides the use of such compounds of formula I-D wherein

W is a) -(C₁-C₄)alkyl- or b) -(C₁-C₄)alkyl-O-; and

A is phenyl optionally substituted with a) -(C₁-C₄)alkyl, b) —CF₃, c) —OCF₃, d) -(C₁-C₄)alkoxy, e) cyclopropyl, f) halo, g) -(C₁-C₄)alkylthio or h) hydroxy.

More particularly, the present invention also provides the use of such compounds of formula I-D wherein

W is a bond; and

A is a) thiazolyl optionally substituted with 1) one or two -methyl or 2) -phenyl optionally substituted with i) -(C₁-C₄)alkyl, ii) —CF₃, iii) —OCF₃ iv) -(C₁-C₄)alkoxy, v) cyclopropyl or vi) halo; or b) phenyl optionally substituted with 1) -(C₁-C₄)alkyl, 2) —CF₃, 3) —OCF₃, 4) -(C₁-C₄)alkoxy, 5) cyclopropyl, 6) halo or 7) -(C₁-C₄)alkylthio.

More particularly, the present invention provides the use of compounds of formula I as recited as examples in the experimental section hereinafter.

Another aspect of the invention is the use of a compound of formula I, in the manufacture of a medicament for the palliative, prophylactic or curative treatment of ruminant disease associated with negative energy balance in ruminants.

Another aspect of the invention is the use of a compound of formula I, in the manufacture of a medicament for the palliative, prophylactic or curative treatment of negative energy balance in ruminants, wherein the excessive accumulation of triglycerides in liver tissue is prevented or alleviated, and/or the excessive elevation of non-esterified fatty acid levels in serum is prevented or alleviated.

Another aspect of the invention is the use of a compound of formula I, in the manufacture of a medicament for the palliative, prophylactic or curative treatment of ruminant disease associated with negative energy balance in ruminants, wherein the excessive accumulation of triglycerides in liver tissue is prevented or alleviated and/or the excessive elevation of non-esterified fatty acid levels in serum is prevented or alleviated.

Preferably, the ruminant disease associated with negative energy balance in ruminants, as mentioned in the aspects of the invention herein, includes one or more diseases selected independently from fatty liver syndrome, dystocia, immune dysfunction, impaired immune function, toxification, primary and secondary ketosis, downer cow syndrome, indigestion, inappetence, retained placenta, displaced abomasum, mastitis, (endo-)-metritis, infertility, low fertility, lameness, subacute rumen acidosis and inadequate nutrient intake associated with stress e.g. heat, poor housing, overcrowding, shipping, dominance or illness. The invention also provides the ability to modify standard dairy cow diet whilst maintaining adequate energy balance.

Even more preferably, the ruminant disease associated with negative energy balance in ruminants, as mentioned in the aspects of the invention herein, includes one or more diseases selected from fatty liver syndrome, primary ketosis, downer cow syndrome, (endo-)-metritis and low fertility.

Another aspect of the invention is the use of a compound of formula I, in the improvement of fertility, including decreased return to service rates, normal oestrus cycling, improved conception rates, and improved foetal viability.

Another aspect of the invention is the use of a compound of formula I, in the manufacture of a medicament for the management of effective homeorhesis to accommodate parturition and lactogenesis.

Another aspect of the invention is the use of a compound of formula I, in the manufacture of a medicament for improving or maintaining the functioning of the ruminant liver and homeostatic signals during the transition period.

In one aspect of the invention, the compound of formula I is administered during the period from 30 days prepartum to 70 days postpartum.

In another aspect of the invention, the compound of formula I is administered prepartum and, optionally, also at parturition.

In yet another aspect of the invention, the compound of formula I is administered postpartum.

In yet another aspect of the invention, the compound of formula I is administered at parturition.

More preferably, the compound of formula I is administered during the period from 3 weeks prepartum to 3 weeks postpartum.

In another aspect of the invention, the compound of formula I is administered up to three times during the first seven days postpartum.

Preferably, the compound of formula I is administered once during the first 24 hours postpartum.

In another aspect of the invention, the compound of formula I is administered prepartum and up to four times postpartum.

In another aspect of the invention, the compound of formula I is administered at parturition and then up to four times postpartum.

Another aspect of the invention is the use of the compound of formula I in the manufacture of a medicament for the palliative, prophylactic or curative treatment of negative energy balance in ruminants, and to increase ruminant milk quality and/or milk yield.

In a preferred aspect of the invention, the milk quality increase is seen in a reduction in the levels of ketone bodies in ruminant milk.

In another aspect of the invention, peak milk yield is increased.

Preferably, the ruminant is a cow or sheep.

In another aspect of the invention, an overall increase in ruminant milk yield is obtained during the 305 days of the bovine lactation period.

In another aspect of the invention, an overall increase in ruminant milk yield is obtained during the first 60 days of the bovine lactation period.

Preferably, the overall increase in ruminant milk yield, or the increase in peak milk yield, or the increase in milk quality, is obtained from a dairy cow.

In another aspect of the invention, the increase in ruminant milk quality and/or milk yield is obtained after administration of a compound of formula I to a healthy ruminant.

In another aspect of the invention, there is provided a compound of formula I, for use in veterinary medicine.

In a preferred aspect of the invention, there is provided a compound of formula I, for use in the palliative, prophylactic or curative treatment of negative energy balance in ruminants.

In an even more preferred aspect of the invention, there is provided a compound of formula I, for use in the palliative, prophylactic or curative treatment of ruminant disease associated with negative energy balance in ruminants, wherein, preferably, the disease is selected from fatty liver syndrome, dystocia, immune dysfunction, impaired immune function, toxification, primary and secondary ketosis, downer cow syndrome, indigestion, inappetence, retained placenta, displaced abomasum, mastitis, (endo-)-metritis, infertility, low, lameness, subacute rumen acidosis and inadequate nutrient intake associated with stress e.g. heat, poor housing, overcrowding, shipping, dominance or illness.

In another aspect of the invention, there is provided a compound of formula I for use in the palliative, prophylactic or curative treatment of negative energy balance in ruminants, and for increasing ruminant milk quantity and/or quality.

In another aspect of the invention, there is provided a kit for the curative, prophylactic or palliative treatment of negative energy balance in ruminants, comprising:
a) a compound of formula I, and
b) optionally, one or more pharmaceutically acceptable carriers, excipients or diluents, and
c) packaging for containing a) and optionally b)

Preferably, the kit is for the palliative, prophylactic or curative treatment of ruminant diseases associated with negative energy balance in ruminants.

More preferably, the kit is for the palliative, prophylactic or curative treatment of fatty liver syndrome, dystocia, immune dysfunction, impaired immune function, toxification, primary and secondary ketosis, downer cow syndrome, indigestion, inappetence, retained placenta, displaced abomasum, mastitis, (endo-)-metritis, infertility, low fertility and lameness.

Even more preferably, the kit further comprises instructions for the curative, prophylactic or palliative treatment of the negative energy balance or ruminant diseases associated with negative energy balance in ruminants.

The “transition period” means from 30 days prepartum to 70 days postpartum

The term “treating”, “treat”, “treats” or “treatment” as used herein includes prophylactic, palliative and curative treatment.

“Negative energy balance” as used herein means that energy via food does not meet the requirements of maintenance and production (milk).

The term “cow” as used herein includes heifer, primiparous and multiparous cow.

“Healthy ruminant” means where the ruminant does not show signs of the following indications: fatty liver syndrome, dystocia, immune dysfunction, impaired immune function, toxification, primary and secondary ketosis, downer cow syndrome, indigestion, inappetence, retained placenta, displaced abomasum, mastitis, (endo-)-metritis, infertility, low fertility and/or lameness.

Milk “quality” as used herein refers to the levels in milk of protein, fat, lactose, somatic cells, and ketone bodies. An increase in milk quality is obtained on an increase in fat, protein or lactose content, or a decrease in somatic cell levels or ketone bodies levels.

An increase in milk yield can mean an increase in milk solids or milk fat or milk protein content, as well as, or instead of, an increase in the volume of milk produced.

“Excessive accumulation of triglycerides” as used herein means greater than the physiological triglyceride content of 10% w/w in liver tissue.

“Excessive elevation of non-esterified fatty acid levels in serum” as used herein means non-esterified fatty acid levels of greater than 800 μmol/L in serum.

Unless otherwise specified, “prepartum” means 3 weeks before calving until the day of calving.

Unless otherwise specified, “postpartum” means from when the newborn is “expelled” from the uterus to 6 weeks after the newborn was expelled from the uterus.

“At parturition” means the 24 hours after the newborn was expelled from the uterus.

“Periparturient” means the period from the beginning of the prepartum period, to the end of the postpartum period.

By “pharmaceutically acceptable” is meant the carrier, diluent, vehicle, excipient, and/or salt must be compatible with the other ingredients of the formulation, and not deleterious to the recipient thereof.

As used herein, “therapeutically effective amount of a compound” means an amount that is effective to exhibit therapeutic or biological activity at the site(s) of activity in a ruminant, without undue adverse side effects (such as undue toxicity, irritation or allergic response), commensurate with a reasonable benefit/risk ratio when used in the manner of the present invention.

The mention of use of compounds in the present invention, shall at all times be understood to include all active forms of such compounds, including, for example, the free form thereof, e.g., the free acid or base form, and also, all prodrugs, polymorphs, hydrates, solvates, tautomers, stereoisomers, e.g., diastereomers and enantiomers, and the like, and all pharmaceutically acceptable salts as described above, unless specifically stated otherwise. It will also be appreciated that the use of suitable active metabolites of such compounds, in any suitable form, are also included herein.

The expression “prodrug” refers to compounds that are drug precursors which following administration release the drug in vivo via some chemical or physiological process (e.g., a prodrug on being brought to the physiological pH or through enzyme action is converted to the desired drug form). Exemplary prodrugs upon cleavage release the corresponding free acid, and such hydrolyzable ester-forming residues of the Formula I compounds include but are not limited to those having a carboxyl moiety wherein the free hydrogen is replaced by (C₁-C₄)alkyl, (C₂-C₇)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Descriptions of exemplary ring(s) for the generic ring descriptions contained in compounds of formula (I) and descriptions of other terms used in formula (I) and in the process sections, including isotopically labelled compounds, are found in US 60/574171 and in WO04/048334, at pages 37-41, which are incorporated herein by reference.

### DETAILED DESCRIPTION OF THE INVENTION

In general the compounds used in the present invention can be made by processes including processes analogous to those known in the chemical arts, and as described in US60/574171 and in WO04/048334, at pages 41-67, which are incorporated herein by reference.

Prodrugs of the compounds of formula I can be prepared according to methods analogous to those known to those skilled in the art, and as described in US60/574171 and in WO04/048334, at pages 68-69, which are incorporated herein by reference.

Some of the formula I compounds used in the present invention or intermediates in their synthesis have asymmetric carbon atoms and therefore are enantiomers or diastereomers. Methods of separation of diasteromeric and enantiomeric mixtures include those well known to those skilled in the art and are further described in US60/574171 and in WO04/048334, at page 84, which is incorporated herein by reference.

Some of the formula I compounds used in the present invention are acidic and they form a salt with a pharmaceutically acceptable cation. Some of the formula I compounds used in the present invention are basic and they form a salt with a pharmaceutically acceptable anion. All such salts are within the scope of the present invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds can be obtained in crystalline form by dissolution in an appropriate solvent(s) such as ethanol, hexanes or water/ethanol mixtures.

Those skilled in the art will recognize that some of the compounds herein can exist in several tautomeric forms. All such tautomeric forms are considered as part of the present invention. For example all enol-keto forms of the compounds of formula I used in the present invention are included in this invention.

In addition, when the formula I compounds used in the present invention form hydrates or solvates they are also within the scope of the present invention.

The formula I compounds for use in the present invention, their prodrugs and the salts of such compounds and prodrugs are all adapted to therapeutic use as agents that activate peroxisome proliferator activator receptor (PPAR) activity in ruminants. Thus, it is believed the compounds for use in the present invention, by activating the PPAR receptor, stimulate transcription of key genes involved in fatty acid oxidation. By virtue of their activity, these agents also reduce plasma levels of triglycerides and NEFA's and prevent accumulation of triglycerides in the liver in ruminants.

The utility of the formula I compounds of the present invention, their prodrugs and the salts of such compounds and prodrugs as agents in the treatment of the above described disease/conditions in ruminants is demonstrated by the activity of the compounds of the present invention in the assays described below.

PPAR FRET Assay

Measurement of coactivator recruitment by a nuclear receptor after receptor-ligand association is a method for evaluating the ability of a ligand to produce a functional response through a nuclear receptor. The PPAR FRET (Fluorescence Resonance Energy Transfer) assay measures the ligand-dependent interaction between nuclear receptor and coactivator. GST/PPAR (α, β, and γ) ligand binding domain (LBD) is labeled with a europium-tagged anti-GST antibody, while an SRC-1 (Sterol Receptor Coactivator-1) synthetic peptide containing an amino terminus long chain biotin molecule is labeled with streptavidin-linked allophycocyanin (APC). Binding of ligand to the PPAR LBD causes a conformational change that allows SRC-1 to bind. Upon SRC-1 binding, the donor FRET molecule (europium) comes in close proximity to the acceptor molecule (APC), resulting in fluorescence energy transfer between donor (337 nm excitation and 620 nm emission) and acceptor (620 nm excitation and 665 nm emission). Increases in the ratio of 665 nm emission to 620 nm emission is a measure of the ability of the ligand-PPAR LBD to recruit SRC-1 synthetic peptide and therefore a measure of the ability of a ligand to produce a functional response through the PPAR receptor.
[1] GST/PPAR LBD Expression. The human PPARα LBD (amino acids 235-507) is fused to the carboxy terminus of glutathione S-transferase (GST) in pGEX-6P-1 (Pharmacia, Piscataway, N.J.). The GST/PPARα LBD fusion protein is expressed in BL21[DE3]pLysS cells using a 50 uM IPTG induction at room temperature for 16 hr (cells induced at an A₆₀₀ of ˜0.6). Fusion protein is purified on glutathione sepharose 4B beads, eluted in 10 mM reduced glutathione, and dialyzed against 1×PBS at 4° C. Fusion protein is quantitated by Bradford assay (M. M. Bradford, Analst. Biochem. 72:248-254; 1976), and stored at −20° C. in 1×PBS containing 40% glycerol and 5 mM DTT.
[2] FRET Assay. The FRET assay reaction mix consists of 1×FRET buffer (50 mM Tris-Cl pH 8.0, 50 mM KCl, 0.1 mg/ml BSA, 1 mM EDTA, and 2 mM DTT) containing 20 nM GST/PPARα LBD, 40 nM of SRC-1 peptide (amino acids 676-700, 5′-long chain biotin-CPSSHSSLTERHKILHRLLQEGSPS-NH₂, purchased from American Peptide Co., Sunnyvale, Calif.), 2 nM of europium-conjugated anti-GST antibody (Wallac, Gaithersburg, Md.), 40 nM of streptavidin-conjugated APC (Wallac), and control and test compounds. The final volume is brought to 100 ul with water and transferred to a black 96-well plate (Microfuor B, Dynex (Chantilly, Va.)). The reaction mixes are incubated for 1 hr at 4° C. and fluorescence is read in Victor 2 plate reader (Wallac). Data is presented as a ratio of the emission at 665 nm to the emission at 615 nm.
   Selectivity Measurements

Transient transfections assay using the HepG2 hepatoma cell line.

HepG2 cells were transiently transfected with an expression plasmids encoding hPPARα, hPPARβ or mPPARγ chimeric receptors and a reporter containing the yeast upstream activating sequence (UAS) upstream of the viral E1B promoter controlling a luciferase reporter gene. In addition, the plasmid pRSVβ-gal was used to control for transfection efficiency. HepG2 cells were grown in DMEM supplemented with 10% FBS and 1 μM non-essential amino acid. On the first day, cells were split into 100 mm dishes at 2.5×10⁶/dish and incubated overnight at 37° C./5% CO₂. On the second day the cells were transiently transfected with plasmid DNA encoding a chimeric receptor, the luciferase reporter gene; and β-gal. For each 100 mm dish, 15 μg of lucifease reporter (PG5E1b) DNA, 15 μg of Gal4-PPAR chimeric receptor DNA, and 1.5 μg of β-gal plasmid DNA were mixed with 1.4 ml of opti-MEM in the tube. 28 μl of LipoFectamine-2000 reagent was added to 1.4 ml of opti-MEM in the tube, and incubate for 5 min at RT. The diluted Lipofectamine-2000 reagent was combined with the DNA mixture, and incubate for 20 min at RT. After fresh medium was added to each 100 mm dish of cells, 2.8 ml of Lipofectamine2000-DNA mixture was added dropwise to the 100 mm dish containing 14 ml of medium, and incubate 37° C. overnight. On day three cells were trypsinized off the100 mm dishes and re-plated on 96 well plates. Cells were plated at 2.5×10⁴ cells per well in 150 μl of media and 50 μl of compound diluted by media was added. The concentrations of reference agents and test compound added were in the range from 50 μM to 50 pM. After addition of compounds, the plates were incubated at 37° C. for 24 hours. Subsequently cells were washed once with 100 μl of PBS, lysed, and processed for measuring luciferase and β-gal activity using Dual-Light luciferase kit from Tropix®, according to the manufacturer's recommendations, on an EG&G Bethold MicroLumat LB96P luminometer. Hep G2-hBeta EC₅₀ values (“EC₅₀β”) and Hep G2-hAlpha EC_{50.} values, (“EC₅₀α”) were obtained using the GraphPad Prism™ program. EC₅₀ is the concentration at which the PPAR mediated transcriptional response reaches one-half of its maximal response.

Negative Energy Balance

To determine negative energy balance, serum concentrations of NEFAs or ketone bodies, or levels of triglycerides in liver tissues, are measured. Higher than ‘normal’ levels of NEFA's and/or triglycerides and/or ketone bodies are indicators of negative energy balance. Levels considered ‘higher than normal’ or ‘excessive’ are:
NEFA's >800 μmol/L in serum.
Triglycerides >10% w/w in liver tissue.
Ketone bodies >1.2 μmol/L in serum.
   Determination of Changes in Blood Non-Esterified Fatty Acid (NEFA) Concentrations and Liver Triglycerides Levels:

Compounds were administered once or several times in the transition period at dose levels predicted to be effective by comparing results of in-vitro receptor affinity tests in laboratory species and pharmacokinetic evaluations in cattle. NEFA levels were determined via standard laboratory methods, for example, using the commercial WAKO NEFA kit (Wako Chemical Co., USA, Dallas, Tex., 994-75409), and liver triglyceride content was determined using the method as described in the literature (J. K. Drackley, J. J. Veenhuizen, M. J. Richard and J. W. Young, J Dairy Sci, 1991, 74, 4254)).

All animals were obtained from a commercial dairy farm approximately thirty days prior to anticipated calving date. The cows were moved into separate building, approximately 10-14 days prior to their anticipated calving dates and switched to the TMR-Close-Up dry diet. Enrolment of animals in the study began approximately 7 days prior to their anticipated calving dates. The animals were moved to the “on-test” pen, weighed and were locked each AM into feed stanchions. At that time, appropriate doses were administered and appropriate blood samples obtained (see table below).

| | | | Pre Partum Dosing | | |
|---|---|---|---|---|---|
| | | | (every other day = | | |
| | | Animals per | eod − beginning | Treatment at | Post Partum Dosing |
| Treatment | Dosage | Treatment | targeted day − 7) | Calving | (eod 4 doses) |
| T01 | — | 9 | X | X | |
| Vehicle Control | | | | | |
| T02 | 0.5 mg/kg | 8 | X | X | |
| Compound Z | | | | | |
| T03 | 0.5 mg/kg | 11 | | X | X |
| Compound | | | | | |
| T04 | 0.5 mg/kg | 9 | | X | |
| Compound Z | | | | | |

As soon as possible post-calving (˜30 minutes) the cow was transferred to the freestall barn for the next scheduled milking (6:00 hrs and 19:00 hrs). Treatments on postpartum animals were administered every other day through day 8. Pre and post-calving NEFA samples were analyzed using the WAKO NEFA-C test kit (#994-75409). Post-calving liver biopsies were performed on all cows on days 5, 10 and 14 post-calving. Tissues were transported on ice and stored frozen at −70° F. At the conclusion of the study, samples were analysed of liver triglyceride levels using the method described by Drackley, J. K. et al. (1991, J Dairy Sci (74):4254-4264).

All animals treated with Compound Z, (3S)-3-[3-(1-Carboxy-1-methyl-ethoxy)-phenyl]-piperidine-1-carboxylic acid 4-trifluoromethyl-benzyl ester, exhibited significantly lower serum NEFA levels from Day 1 (after calving) until Day 6 of the study as compared to controls. In addition, animals in treatment group T03 exhibited significantly lower serum NEFA levels compared to controls at all timepoints. All treatment regimens significantly lowered liver triglyceride levels compared to placebo at all time points measured (Days 5, 10 and 14 postcalving).

Ketone Bodies

Levels of ketone bodies in serum can be measured by standard methods well known to the person skilled in the art, for example, by using the commercially available kits for this purpose, including Sigma BHBA kit of order number 310-A.

Milk Content:

Machines to assay for milk protein, fat, or lactose content are commercially available (MilkoScan™ 50, MilkoScan™ 4000, MilkoScan™ FT 6000 available from Foss Group). Machines to assay for somatic cell content are also commercially available (Fossomatic™ FC, Fossomatic™ Minor available from Foss Group).

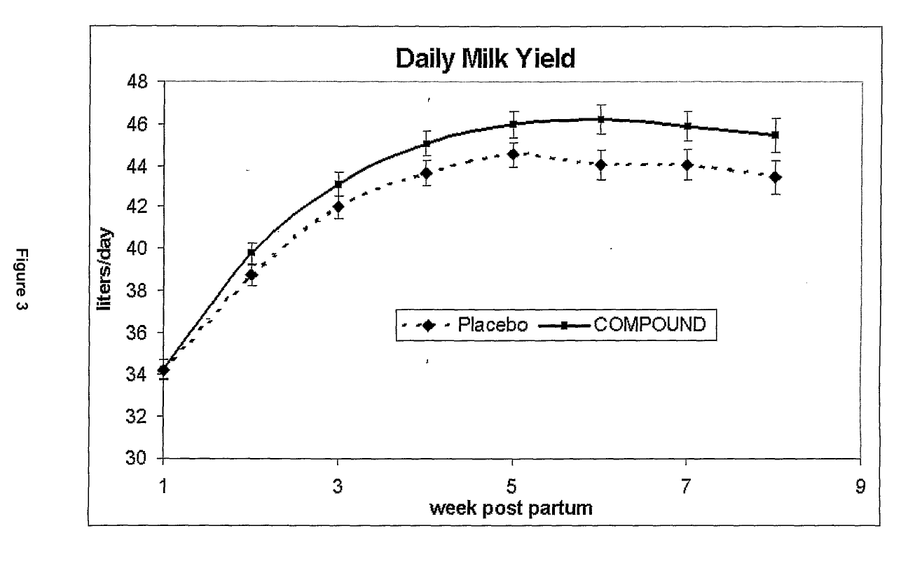

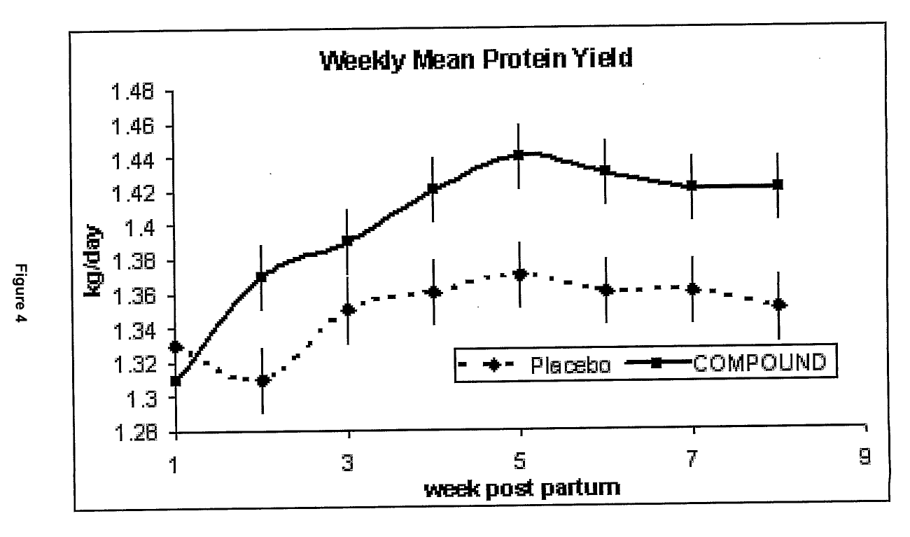

One hundred twenty four pregnant, non-lactating Holstein cows were allocated to two treatment groups (placebo and COMPOUND at approximately 0.5 mg/kg). Animals were allowed to calve, treated by subcutaneous injection on the day of calving and on day five post-calving. Disease events and daily milk production were recorded for the following sixty days. The average daily milk yield in the treated cows was increased from 41.8 to 43.2 kg/day (p=0.052). There was also a significant beneficial effect on milk quality (increased protein and lactose yield, decreased somatic cell count). Results are shown in the FIG. 3 and FIG. 4, where COMPOUND represents Compound Z.

Compounds used in this invention may be administered alone or in combination with one or more other compounds of the invention or in combination with one or more other drugs (or as any combination thereof).

For example, compounds of this invention can also be mixed with one or more biologically active compounds or agents selected from sedatives, analgesics, antiinflammatories, analeptics, antibacterials, antidiarrhoeals, anti-endotoxin, antifungals, respiratory stimulants, corticosteroids, diuretics, parasiticides, electrolyte preparations and nutritional supplements, growth promoters, hormones, and metabolic disease treatments, giving an even broader spectrum of veterinary or agricultural utility.

Examples of suitable active compounds or agents are found below:
Rumen Amylase and or glucosidae inhibitors, e.g. acarbose
Sedative: alpha adrenergic agonists, e.g. xylazine,
Analgesics and antiinflammatories: Lignocaine, Procaine, flunixin, oxytetracycline, ketoprofen, meloxicam and carprofen.
Analeptics: Etamiphylline, Doxapram, Diprenorphine, Hyoscine, Ketoprofen, Meloxicam, Pethidine, Xylazine and Butorphanol,
Antibacterials: Chlortetracycline, Tylosin, Amoxycillin, Ampicillin, Aproamycin, Cefquinome, Cephalexin, Clavulanic acid, Florfenicol, Danofloxacin, Enrofloxacin, Marbofloxacin, Framycetin, Procaine penicillin, procaine benzylpenicillin, Benzathine penicillin, sulfadoxine, Trimethoprim, sulphadimidine, baquiloprim, streptomycin, dihydrostreptomycin, sulphamethoxypyridazine, sulphamethoxypuridazine, oxytetracycline, flunixin, tilmicosin, cloxacillin, ethyromycin, neomycin, nafcillin, Aureomycin, lineomycin, cefoperazone, cephalonium, oxytetracycline, formosulphathiazole, sulphadiazine and zinc.
Antidiarrhoeals: Hyoscine, Dipyrone, charcoal, attapulgite, kaolin, Isphaghula husk,
Anti-endotoxins: Flunixin, ketoprofen,
Antifungals: Enilconazole, Natamycin,
Respiratory stimulants: florfenicol,
Corticosteroids: dexamethasone, betamethasone,
Diuretics: frusemide,
Parasiticides—amitraz, deltamethrin, moxidectin, doramectin, alpha cypermethrin, fenvalerate, eprinomectin, permethrin, ivermectin, abamectin, ricobendazole, levamisole, febantel, triclabendazole, fenbendazole, albendazole, netobimin, oxfenazole, oxyclozanide, nitroxynil, morantel,
Electrolyte preparations and nutritional supplements: dextrose, lactose, propylene glycol, whey, glucose, glycine, calcium, cobalt, copper, iodine, iron, magnesium, manganese, phosphorous, selenium, zinc, Biotin, vitamin B₁₂, Vitamin E, and other vitamins,
Growth Promoters: monensin, flavophospholipol, bambermycin, salinomycin, tylosin,
Hormones: chorionic gonadotrophin, serum gonadotrophin, atropine, melatonin, oxytocin, dinoprost, cloprostenol, etiproston, luprostiol, buserelin, oestradiol, progesterone, and bovine somatotropin,
Metabolic Disease Treatments: calcium gluconate, calcium borogluconate, propylene glycol, magnesium sulphate,

Compounds of this invention can also be mixed with one or more biologically active compounds or agents selected from antiprotozoals such as imidocarb, bloat remedies such as dimethicone and poloxalene, and probiotics such as Lactobacilli and streptococcus.

Other compounds which may be mixed with compounds for use in the invention include rumen protected choline; DCAD; amino acids e.g. glutamine, lysine, serine, methionine, alanine, aspartamine; probiotics e.g. Propionibacterium, Teichomycin A2; yeasts; glucocorticoids: glucose precursors e.g. glucagon, propylene glycol, propionic acid, propyl esters, propyl alcohol, lactose, glycerol, pyruvate; vegetable oils, e.g. safflower; fish oils; unsaturated fatty acids e.g. CLA; algae extracts (to increase omega fatty acids); plant sterols e.g. ergosterol; alpha-ketoisocaproate; vitamin D; calcium and magnesium salts; miscellaneous branded treatments: Reassure, Rally, MEGALAC, Fermenten, Rumensin crc bolus; and miscellaneous antiinflammatory agents: prednisolone; antibiotic ionophores e.g. nigericin, tetronasin; antibiotics: cefamezin and metronidazole.

As a preferred feature of the present invention, alpha amylase and alpha glucosidase inhibitors e.g. acarbose, may be combined with a PPAR agonist compound described herein, particularly an exemplified or preferred compound, for use according to the present invention.

Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

With respect to their use in ruminants, the compounds may be administered alone or in a formulation appropriate to the specific use envisaged. The routes and methods of administration of formulations for use according to the present invention, which were described in full in the priority filing for the present application, are also published in US 60/574171 and in WO04/048334, at pages 94-97, which are incorporated herein by reference.

Such formulations are prepared in a conventional manner in accordance with standard veterinary practice.

These formulations will vary with regard to the weight of active compound contained therein, depending on the species of host animal to be treated, the severity and type of infection and the body weight of the host. For parenteral, topical and oral administration, typical dose ranges of the active ingredient are 0.05 to 5 mg per kg of body weight of the animal. Preferably the range is 0.01 to 1 mg per kg.

As an alternative the compounds may be administered to a ruiminant with the drinking water or feedstuff and for this purpose a concentrated feed additive or premix may be prepared for mixing with the normal animal feed or drink.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to ruminants, the total daily dose of the compounds of the invention is typically in the range 0.05 mg/kg to 5 mg/kg depending, of course, on the mode of administration. For example, oral administration may require a total daily dose of from 0.05 mg/kg to 5 mg/kg, while an intravenous dose may only require from 0.01 mg/kg to 1 mg/kg. The total daily dose may be administered in single or divided doses. The veterinarian will readily be able to determine doses for individual ruminants according to age, weight and need.

### FORMULATION EXAMPLES

In the formulations which follow, “active ingredient” means a compound used in the present invention.

### Formulation 1

### Solution for Parenteral Administration

Solution of active ingredient will be prepared as follows:

| Ingredient | Quantity (mg/5 ml) |
|---|---|
| Active ingredient | 1-750 |
| Potassium hydroxide | 0-75 |
| Sodium hydroxide | 0-75 |
| Sodium dihydrogen phosphate | 0-50 |
| Disodium hydrogen phosphate | 0-100 |
| PVP | 0-50 |
| Methyl Paraben | 0-40 |
| Water | Up to 5 ml |

Or

### Formulation 2

### Solution for Parenteral Administration

Solution of active ingredient will be prepared as follows:

| Ingredient | Quantity (mg/5 ml) |
|---|---|
| Active ingredient | 1-750 |
| Sodium dihydrogen phosphate | 0-50 |
| Disodium hydrogen phosphate | 0-100 |
| Methyl Paraben | 0-40 |
| Water | Up to 5 ml |

Or

### Formulation 3

### Solution for Parenteral Administration

Solution of active ingredient will be prepared as follows:

| Ingredient | Quantity (mg/5 ml) |
|---|---|
| Active ingredient | 1-500 |
| Hydroxy propyl β-cyclodextrin | 10-4000 |
| Methyl Paraben | 0-40 |
| Water | Up to 5 ml |

### Formulation 4

### Solution for Subcutaneous Administration

Solution of active ingredient will be prepared as follows:

| Ingredient | Quantity (mg) |
|---|---|
| Active ingredient | 1-500 |
| Glycerol Formal | 100-10000 |

### Formulation 5

### Gelatin Capsules

Hard gelatin capsules are prepared using the following:

| Ingredient | Quantity (mg/capsule) |
|---|---|
| Active ingredient | 1-500 |
| Starch, NF | 0-1000 |
| Starch flowable powder | 0-250 |
| Silicone fluid 350 centistokes | 0-45 |

### Formulation 6

### Tablets

A Tablet Formulation is prepared using the ingredients below:

| Ingredient | Quantity (mg/tablet) |
|---|---|
| Active ingredient | 0.25-500 |
| Cellulose, microcrystalline | 100-1000 |
| Silicon dioxide, fumed | 10-1000 |
| Stearate acid | 5-50 |

The components are blended and compressed to form tablets.

Alternatively, tablets each containing 1-500 mg of active ingredients are made up as follows:

### Formulation 7

### Tablets

| Ingredient | Quantity (mg/tablet) |
|---|---|
| Active ingredient | 1-500 |
| Starch | 45-200 |
| Cellulose, microcrystalline | 35-100 |
| Polyvinylpyrrolidone (as 10% solution in water) | 4-20 |
| Sodium carboxymethyl cellulose | 4.5 |
| Magnesium stearate | 0.5-2 |
| Talc | 1-5 |

The active ingredients, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50°-60° C. and passed through a No. 18 mesh U.S. sieve. The sodium carboxymethyl starch, magnesium stearate, and talc, previously passed through a No. 60 U.S. sieve, are then added to the granules which, after mixing, are compressed on a tablet machine to yield tablets.

Suspensions each containing 1-750 mg of active ingredient per 5 ml dose are made as follows:

### Formulation 4

### Suspensions

| Ingredient | Quantity (mg/5 ml) | |
|---|---|---|
| Active ingredient | 1-750 | mg |
| Sodium carboxymethyl cellulose | 50 | mg |
| Syrup | 1.25 | mg |
| Benzoic acid solution | 0.10 | mL |
| Flavor | q.v. | |
| Color | q.v. | |
| Purified Water to | 5 | mL |

The active ingredient is passed through a No. 45 mesh U.S. sieve and mixed with the sodium carboxymethyl cellulose and syrup to form a smooth paste. The benzoic acid solution, flavor, and color are diluted with some of the water and added, with stirring. Sufficient water is then added to produce the required volume.

### GENERAL EXPERIMENTAL PROCEDURES

For the sake of brevity, the preparation of the Preparations and Examples given below, which were described in full in the priority filing for the present application, are also published in US 60/574171 and in WO04/048334. All the experimental details are incorporated herein by reference.
Preparation 1: 3-(3-Methoxyphenyl)-1H-piperidine
Preparation 2: 2-methyl-2-(3-piperidin-3-yl-phenoxy)-propionic acid alkyl esters
Preparation 3 Resolution of 2-methyl-2-(3-piperidin-3-yl-phenoxy)-propionic acid alkyl esters
Example 1 2-: (3-{1-[(4-Isopropyl-phenyl)-acetyl]-piperidin-3-yl}-phenoxy)-2-methyl-propionic acid benzyl ester
Example 1-12-: (3-{1-[(3-Methoxy-phenyl)-acetyl]-piperidin-3-yl}-phenoxy)-2-methyl-propionic acid
Example 1-22-: (3-{1-[(4-Methoxy-phenyl)-acetyl]-piperidin-3-yl}-phenoxy)-2-methyl-propionic acid
Example 1-32: (3-{1-[(4-Fluoro-phenyl)-acetyl]-piperidin-3-yl}-phenoxy)-2-methyl-propionic acid
Example 1-4: 2-(3-{1-[(4-Hydroxy-phenyl)-acetyl]-piperidin-3-yl}-phenoxy)-2-methyl-propionic acid
Example 1-5: 2-{3-[1-(4-Isopropyl-benzoyl)-piperidin-3-yl]-phenoxy}-2-methyl-propionic acid
Example 1-6: 2-(3-{1-[(2,4-Dimethoxy-phenyl)-acetyl]-piperidin-3-yl}-phenoxy)-2-methyl-propionic acid
Example 1-7: 2-Methyl-2-(3-{1-[(4-trifluoromethyl-phenyl)-acetyl]-piperidin-3-yl}-phenoxy)-propionic acid
Example 1-8: 2-(3-{1-[3-(3-Methoxy-phenyl)-propionyl]-piperidin-3-yl}-phenoxy)-2-methyl-propionic acid
Example 1-9: 2-Methyl-2-{3-[1-(pyridin-2-yl-acetyl)-piperidin-3-yl]-phenoxy}-propionic acid
Example 1-10: 2-Methyl-2-{3-[1-(pyridin-3-yl-acetyl)-piperidin-3-yl]-phenoxy}-propionic acid
Example 1-11: 2-Methyl-2-{3-[1-(pyridin-4-yl-acetyl)-piperidin-3-yl]-phenoxy}-propionic acid
Example 1-12: 2-[3-(1-Cyclohexylacetyl-piperidin-3-yl)-phenoxy]-2-methyl-propionic acid
Example 1-13: (S)-2-(3-{1-[(4-Isopropyl-phenyl)-acetyl]-piperidin-3-yl}-phenoxy)-2-methyl-propionic acid
Example 1-14: (R)-2-(3-{1-[(4-Isopropyl-phenyl)-acetyl]-piperidin-3-yl}-phenoxy)-2-methyl-propionic acid
Example 1-15: 2-[3-(1-Isobutyryl-piperidin-3-yl)-phenoxy]-2-methyl-propionic acid
Example 1-16: 2-Methyl-2-[3-(1-phenylacetyl-piperidin-3-yl)-phenoxy]-propionic acid
Example 1-17: 2-Methyl-2-{3-[1-(3-phenyl-propionyl)-piperidin-3-yl]-phenoxy}-propionic acid
Example 1-18: 2-Methyl-2-[3-(1-m-tolylacetyl-piperidin-3-yl)-phenoxy]-propionic acid
Example 1-19: 2-Methyl-2-{3-[1-(pyridine-2-carbonyl)-piperidin-3-yl]-phenoxy}-propionic acid
Example 1-20: 2-Methyl-2-{3-[1-(pyridine-3-carbonyl)-piperidin-3-yl]-phenoxy}-propionic acid
Example 1-21: 2-[3-(1-Benzoyl-piperidin-3-yl)-phenoxy]-2-methyl-propionic acid
Example 1-22: 2-(3-{1-[(3-Fluoro-phenyl)-acetyl]-piperidin-3-yl}-phenoxy)-2-methyl-propionic acid
Example 1-23: 2-(3-{1-[(3-Chloro-phenyl)-acetyl]-piperidin-3-yl}-phenoxy)-2-methyl-propionic acid
Example 1-24: 2-(3-{1-[(4-Chloro-phenyl)-acetyl]-piperidin-3-yl}-phenoxy)-2-methyl-propionic acid
Example 1-25: 2-Methyl-2-(3-{1-[(4-trifluoromethoxy-phenyl)-acetyl]-piperidin-3-yl}-phenoxy)-propionic acid
Example 1-26: 2-Methyl-2-{3-[1-(3-piperidin-1-yl-propionyl)-piperidin-3-yl]-phenoxy}-propionic acid
Example 1-27: 2-Methyl-2-{3-[1-(3-methyl-butyryl)-piperidin-3-yl]-phenoxy}-propionic acid
Example 1-28: 2-(3-{1-[(4-Ethoxy-phenyl)-acetyl]-piperidin-3-yl}-phenoxy)-2-methyl-propionic acid
Example 1-29: 2-(3-{1-[(2-Methoxy-phenyl)-acetyl]-piperidin-3-yl}-phenoxy)-2-methyl-propionic acid
Example 1-30: 2-Methyl-2-[3-(1-o-tolylacetyl-piperidin-3-yl)-phenoxy]-propionic acid
Example 1-31: 2-Methyl-2-[3-(1-p-tolylacetyl-piperidin-3-yl)-phenoxy]-propionic acid
Example 1-32: 2-(3-{1-[(3,5-Dimethoxy-phenyl)-acetyl]-piperidin-3-yl}-phenoxy)-2-methyl-propionic acid
Example 1-33: 2-Methyl-2-(3-{1-[(3-trifluoromethyl-phenyl)-acetyl]-piperidin-3-yl}-phenoxy)-propionic acid
Example 1-34: 2-(3-{1-[(3,5-Bis-trifluoromethyl-phenyl)-acetyl]-piperidin-3-yl}-phenoxy)-2-methyl-propionic acid
Example 1-35: 2-Methyl-2-(3-{1-[(3-trifluoromethoxy-phenyl)-acetyl]-piperidin-3-yl}-phenoxy)-propionic acid
Example 1-36: 2-Methyl-2-(3-{1-[3-(3-trifluoromethoxy-phenyl)-propionyl]-piperidin-3-yl}-phenoxy)-propionic acid
Example 1-37: 2-Methyl-2-{3-[1-(piperidin-1-yl-acetyl)-piperidin-3-yl]-phenoxy}-propionic acid
Example 1-38: 2-Methyl-2-{3-[1-(morpholin-4-yl-acetyl)-piperidin-3-yl]-phenoxy}-propionic acid
Example 1-39: 2-Methyl-2-{3-[1-(piperazin-1-yl-acetyl)-piperidin-3-yl]-phenoxy}-propionic acid
Example 1-40: 2-(3-{1-[(1H-Benzoimidazol-2-yl)-acetyl]-piperidin-3-yl}-phenoxy)-2-methyl-propionic acid
Example 1-41: 2-{3-[1-(Benzo[1,3]dioxol-5-yl-acetyl)-piperidin-3-yl]-phenoxy}-2-methyl-propionic acid
Example 1-42: 2-(3-{1-[(2-Hydroxy-phenyl)-acetyl]-piperidin-3-yl}-phenoxy)-2-methyl-propionic acid
Example 1-43: 2-(3-{1-[(4-tert-Butyl-phenyl)-acetyl]-piperidin-3-yl}-phenoxy)-2-methyl-propionic acid
Example 1-44: 2-(3-{1-[(4-Ethyl-phenyl)-acetyl]-piperidin-3-yl}-phenoxy)-2-methyl-propionic acid
Example 1-45: 2-{3-[1-(4-Isobutyl-benzoyl)-piperidin-3-yl]-phenoxy}-2-methyl-propionic acid
Example 1-46: 2-(3-{1-[(4-Isobutyl-phenyl)-acetyl]-piperidin-3-yl}-phenoxy)-2-methyl-prop ionic acid
Example 1-47: 2-Methyl-2-(3-{1-[4-(2,2,2-trifluoro-1-hydroxy-1-trifluoromethyl-ethyl)-benzoyl]-piperidin-3-yl}-phenoxy)-propionic acid
Example 1-48: (S)-2-(3-{1-[(4-tert-Butyl-phenyl)-acetyl]-piperidin-3-yl}-phenoxy)-2-methyl-propionic acid
Example 1-49: (S)-2-Methyl-2-(3-{1-[(4-trifluoromethoxy-phenyl)-acetyl]-piperidin-3-yl}-phenoxy)-propionic acid
Example 1-50: (R)-2-Methyl-2-(3-{1-[(4-trifluoromethoxy-phenyl)-acetyl]-piperidin-3-yl}-phenoxy)-propionic acid
Example 1-51: (R)-2-(3-{1-[(4-tert-Butyl-phenyl)-acetyl]-piperidin-3-yl}-phenoxy)-2-methyl-propionic acid
Example 1-52: (S)-2-(3-{1-[(4-Cyclohexyl-phenyl)-acetyl]-piperidin-3-yl}-phenoxy)-2-methyl-propionic acid
Example 1-53: (S)-2-(3-{1-[(4-Methanesulfonyl-phenyl)-acetyl]-piperidin-3-yl}-phenoxy)-2-methyl-propionic acid
Example 1-54: (S)-2-{3-[1-(Biphenyl-4-yl-acetyl)-piperidin-3-yl]-phenoxy}-2-methyl-propionic acid
Example 1-55: (S)-2-Methyl-2-{3-[1-(naphthalen-2-yl-acetyl)-piperidin-3-yl]-phenoxy}-propionic acid
Example 1-56: (S)-2-Methyl-2-(3-{1-[4-methyl-2-(4-trifluoromethyl-phenyl)-thiazole-5-carbonyl]-piperidin-3-yl}-phenoxy)-propionic acid
Example 1-57: (S)-2-Methyl-2-{3-[1-(naphthalen-1-yl-acetyl)-piperidin-3-yl]-phenoxy}-propionic acid
Example 1-58: (S)-2-Methyl-2-(3-{1-[(4-trifluoromethyl-phenyl)-acetyl]-piperidin-3-yl}-phenoxy)-propionic acid
Example 1-59: 2-(4-{1-[(4-Isopropyl-phenyl)-acetyl]-piperidin-3-yl}-phenoxy)-2-methyl-propionic acid
Example 1-60: 2-Methyl-2-(4-{1-[(4-trifluoromethyl-phenyl)-acetyl]-piperidin-3-yl}-phenoxy)-propionic acid
Example 1-61: 2-{4-[1-(4-Isopropyl-benzoyl)-piperidin-3-yl]-phenoxy}-2-methyl-propionic acid
Example 1-62: 2-Methyl-2-{4-[1-(pyridin-2-yl-acetyl)-piperidin-3-yl]-phenoxy}-propionic acid
Example 1-63: 2-(4-{1-[3-(4-isopropyl-phenyl)-propionyl]-piperidin-3-yl}-phenoxy)-2-methyl-propionic acid
Example 1-64: (3-{1-[(4-Isopropyl-phenyl)-acetyl]-piperidin-3-yl}-phenoxy)-acetic acid
Example 2: 2-(3-{1-[(4-Isopropyl-phenoxy)-acetyl]-piperidin-3-yl}-phenoxy)-2-methyl-propionic acid
Example 2-1: 2-(3-{1-[2-(4-Isopropyl-phenoxy)-2-methyl-propionyl]-piperidin-3-yl}-phenoxy)-2-methyl-propionic acid
Example 2-2: 2-Methyl-2-(3-{1-[(4-trifluoromethoxy-phenoxy)-acetyl]-piperidin-3-yl}-phenoxy)-propionic acid
Example 2-3: (S)-2-(3-{1-[(4-Isopropyl-phenoxy)-acetyl]-piperidin-3-yl}-phenoxy)-2-methyl-propionic acid
Example 2-4: (R)-2-(3-{1-[(4-Isopropyl-phenoxy)-acetyl]-piperidin-3-yl}-phenoxy)-2-methyl-propionic acid
Example 2-5: (S)-2-Methyl-2-(3-{1-[(4-trifluoromethoxy-phenoxy)-acetyl]-piperidin-3-yl}-phenoxy)-propionic acid
Example 2-6: (R)-2-Methyl-2-(3-{1-[(4-trifluoromethoxy-phenoxy)-acetyl]-piperidin-3-yl}-phenoxy)-propionic acid
Example 2-7: 2-(3-{1-[(3-Isopropyl-phenoxy)-acetyl]-piperidin-3-yl}-phenoxy)-2-methyl-propionic acid
Example 2-8: 2-(3-{1-[(4-tert-Butyl-phenoxy)-acetyl]-piperidin-3-yl}-phenoxy)-2-methyl-propionic acid
Example 2-9: 2-Methyl-2-[3-(1-m-tolyloxyacetyl-piperidin-3-yl)-phenoxy]-propionic acid
Example 2-10: 2-Methyl-2-(3-{1-[(3-trifluoromethyl-phenoxy)-acetyl]-piperidin-3-yl}-phenoxy)-propionic acid
Example 2-11: (S)-2-(3-{1-[(3-isopropyl-phenoxy)-acetyl]-piperidin-3-yl}-phenoxy)-2-methyl-propionic acid
Example 3: 2-(3-{1-[3-(4-Isopropyl-phenyl)-propionyl]-piperidin-3-yl}-phenoxy)-2-methyl-propionic acid
Example 3-1: 2-Methyl-2-(3-{1-[3-(4-trifluoromethyl-phenyl)-propionyl]-piperidin-3-yl}-phenoxy)-propionic acid
Example 3-2: 2-Methyl-2-(3-{1-[3-(4-trifluoromethoxy-phenyl)-propionyl]-piperidin-3-yl}-phenoxy)-propionic acid
Example 4: 3-[3-(1-Carboxy-1-methyl-ethoxy)-phenyl]-piperidine-1-carboxylic acid 4-isopropyl-phenyl ester
Example 4-1: 3-[3-(1-Carboxy-1-methyl-ethoxy)-phenyl]-piperidine-1-carboxylic acid 3-isopropyl-phenyl ester
Example 4-2: 3-[3-(1-Carboxy-1-methyl-ethoxy)-phenyl]-piperidine-1-carboxylic acid 4-tert-butyl-phenyl ester
Example 4-3: (R)-3-[3-(1-Carboxy-1-methyl-ethoxy)-phenyl]-piperidine-1-carboxylic acid 4-isopropyl-phenyl ester
Example 4-4: (S)-3-[3-(1-Carboxy-1-methyl-ethoxy)-phenyl]-piperidine-1-carboxylic acid 4-isopropyl-phenyl ester
Example 5: 3-[3-(1-carboxy-1-methyl-ethoxy)-phenyl]-piperidine-1-carboxylic acid 4-isopropyl-benzyl ester
Example 5-1: 3-[3-(1-Carboxy-1-methyl-ethoxy)-phenyl]-piperidine-1-carboxylic acid 4-trifluoromethyl-benzyl ester
Example 5-2: (R)-3-[3-(1-Carboxy-1-methyl-ethoxy)-phenyl]-piperidine-1-carboxylic acid 4-isopropyl-benzyl ester
Example 5-3: (S)-3-[3-(1-Carboxy-1-methyl-ethoxy)-phenyl]-piperidine-1-carboxylic acid 4-isopropyl-benzyl ester
Example 5-4: (S)-3-[3-(1-Carboxy-1-methyl-ethoxy)-phenyl]-piperidine-1-carboxylic acid 4-cyclohexyl-benzyl ester
Example 5-5: (S)-3-[3-(1-Carboxy-1-methyl-ethoxy)-phenyl]-piperidine-1-carboxylic acid 4-ethyl-benzyl ester
Example 5-6: (S)-3-[3-(1-Carboxy-1-methyl-ethoxy)-phenyl]-piperidine-1-carboxylic acid 3-trifluoromethyl-benzyl ester
Example 5-7: (S)-3-[3-(1-Carboxy-1-methyl-ethoxy)-phenyl]-piperidine-1-carboxylic acid 4-trifluoromethoxy-benzyl ester
Example 5-8: (S)-3-[3-(1-Carboxy-1-methyl-ethoxy)-phenyl]-piperidine-1-carboxylic acid benzyl ester
Example 5-9: (S)-3-[3-(1-Carboxy-1-methyl-ethoxy)-phenyl]-piperidine-1-carboxylic acid 4-fluoro-benzyl ester
Example 5-10: (S)-3-[3-(1-Carboxy-1-methyl-ethoxy)-phenyl]-piperidine-1-carboxylic acid 4-fluoro-3-trifluoromethyl-benzyl ester
Example 5-11: (S)-3-[3-(1-Carboxy-1-methyl-ethoxy)-phenyl]-piperidine-1-carboxylic acid 3-fluoro-4-trifluoromethyl-benzyl ester
Example 5-12: (S)-3-[3-(1-Carboxy-1-methyl-ethoxy)-phenyl]-piperidine-1-carboxylic acid 3-trifluoromethoxy-benzyl ester
Example 6: 3-[3-(1-carboxy-1-methyl-ethoxy)-phenyl]-piperidine-1-carboxylic acid 4-isopropyl-benzyl ester
Example 6-1: (3S)-3-[3-(1-Carboxy-1-methyl-ethoxy)-phenyl]-piperidine-1-carboxylic acid 4-trifluoromethyl-benzyl ester
Example 6-2: 3-[3-(1-Carboxy-1-methyl-ethoxy)-phenyl]-piperidine-1-carboxylic acid 4-cyclopropyl-benzyl ester
Example 7: (S)-3-[3-(1-carboxy-1-methyl-ethoxy)-phenyl]-piperidine-1-carboxylic acid methyl ester
Example 7-1: (S)-3-[3-(1-Carboxy-1-methyl-ethoxy)-phenyl]-piperidine-1-carboxylic acid 2-methoxy-ethyl ester
Example 7-2: (S)-3-[3-(1-Carboxy-1-methyl-ethoxy)-phenyl]-piperidine-1-carboxylic acid isopropyl ester
Example 7-3: (S)-3-[3-(1-Carboxy-1-methyl-ethoxy)-phenyl]-piperidine-1-carboxylic acid ethyl ester
Example 7-4: (S)-3-[3-(1-Carboxy-1-methyl-ethoxy)-phenyl]-piperidine-1-carboxylic acid isobutyl ester
Example 7-5: (S)-3-[3-(1-Carboxy-1-methyl-ethoxy)-phenyl]-piperidine-1-carboxylic acid cyclohexylmethyl ester
Example 8: 2-methyl-2-{3-[1-(4-trifluoromethyl-benzylcarbamoyl)-piperidin-3-yl]-phenoxy}-propionic acid
Examples 8-1 to 8-6 were prepared from analogous starting materials using methods analogous to those described in Example 8.
Example 8-1: 2-{3-[1-(4-isopropyl-benzylcarbamoyl)-piperidin-3-yl]-phenoxy}-2-methyl-propionic acid
Example 8-2: 2-Methyl-2-{3-[1-(4-trifluoromethoxy-benzylcarbamoyl)-piperidin-3-yl]-phenoxy}-propionic acid
Example 8-3: (S)-2-Methyl-2-{3-[1-(4-trifluoromethoxy-benzylcarbamoyl)-piperidin-3-yl]-phenoxy}-propionic acid
Example 8-4: (S)-2-{3-[1-(4-Isopropyl-benzylcarbamoyl)-piperidin-3-yl]-phenoxyl}-2-methyl-propionic acid
Example 8-5: (S)-2-{3-[1-(Cyclohexylmethyl-carbamoyl)-piperidin-3-yl]-phenoxy}-2-methyl-propionic acid
Example 8-6: 2-{3-[1-(4-Isopropyl-phenylcarbamoyl)-piperidin-3-yl]-phenoxy}-2-methyl-propionic acid
Example 9: (R)-3-(3-carboxy-4-methyl-phenyl)-piperidine-1-carboxylic acid 4-trifluoromethyl-benzyl ester
Example 9-1: (R)-2-methyl-5-{1-[4-methyl-2-(4-trifluoromethyl-phenyl)-thiazole-5-carbonyl]-piperidin-3-yl}-benzoic acid
Example 9-2: (S)-2-methyl-5-{1-[4-methyl-2-(4-trifluoromethyl-phenyl)-thiazole-5-carbonyl]-piperidin-3-yl}-benzoic acid
Example 9-3: 2-methyl-5-{1-[4-methyl-2-(4-trifluoromethyl-phenyl)-thiazole-5-carbonyl]-piperidin-3-yl}-benzoic acid
Example 9-4: (S)-3-(3-carboxy-4-methyl-phenyl)-piperidine-1-carboxylic acid 4-trifluoromethyl-benzyl ester
Example 9-5: 3-(3-carboxy-4-methyl-phenyl)-piperidine-1-carboxylic acid 4-trifluoromethyl-benzyl ester
Example 9-6: 2-Methyl-5-{1-[(4-trifluoromethoxy-phenyl)-acetyl]-piperidin-3-yl}-benzoic acid
Example 9-7: 5-{1-[(4-Isopropyl-phenyl)-acetyl]-piperidin-3-yl}-2-methyl-benzoic acid
Example 9-8: 2-Methyl-5-{1-[(4-trifluoromethyl-phenyl)-acetyl]-piperidin-3-yl}-benzoic acid
Example 9-9: 2-Methyl-5-{1-[3-(4-trifluoromethyl-phenyl)-acryloyl]-piperidin-3-yl}-benzoic acid
Example 9-10: 5-{1-[3-(4-Isopropyl-phenyl)-acryloyl]-piperidin-3-yl}-2-methyl-benzoic acid
Example 9-11: 2-Methyl-5-{1-[3-(4-trifluoromethyl-phenyl)-propionyl]-piperidin-3-yl}-benzoic acid
Example 9-12: 5-{1-[3-(4-Isopropyl-phenyl)-propionyl]-piperidin-3-yl}-2-methyl-benzoic acid
Example 9-13: 3-(3-Carboxy-4-methyl-phenyl)-piperidine-1-carboxylic acid 4-isopropyl-benzyl ester
Example 9-14: (R)-2-Methyl-5-[1-(4-trifluoromethyl-benzylcarbamoyl)-piperidin-3-yl]-benzoic acid
Example 9-15: (S)-2-Methyl-5-[1-(4-trifluoromethyl-benzylcarbamoyl)-piperidin-3-yl]-benzoic acid
Example 9-16: (R)-3-(3-Carboxy-4-methyl-phenyl)-piperidine-1-carboxylic acid 2-(4-trifluoromethyl-phenyl)-ethyl ester
Example 9-17: 2-Methyl-4-[1-(4-trifluoromethyl-benzoyl)-piperidin-3-yl]-benzoic acid
Example 9-18: 2-Methyl-4-{1-[(4-trifluoromethyl-phenyl)-acetyl]-piperidin-3-yl}-benzoic acid
Example 9-19: 2-Methyl-4-{1-[3-(4-trifluoromethyl-phenyl)-acryloyl]-piperidin-3-yl}-benzoic acid
Example 9-20: 2-Methyl-4-{1-[4-methyl-2-(4-trifluoromethyl-phenyl)-thiazole-5-carbonyl]-piperidin-3-yl}-benzoic acid
Example 9-21: 3-(4-Carboxy-3-methyl-phenyl)-piperidine-1-carboxylic acid 4-trifluoromethyl-benzyl ester
Example 9-22: 4-[1-(4-Isopropyl-benzoyl)-piperidin-3-yl]-2-methyl-benzoic acid
Example 9-23: 4-{1-[(4-Isopropyl-phenyl)-acetyl]-piperidin-3-yl}-2-methyl-benzoic acid
Example 9-24: 4-{1-[3-(4-Isopropyl-phenyl)-acryloyl]-piperidin-3-yl}-2-methyl-benzoic acid
Example 9-25: 3-(4-Carboxy-3-methyl-phenyl)-piperidine-1-carboxylic acid 4-isopropyl-benzyl ester
Example 9-26: 2-Methyl-4-{1-[3-(4-trifluoromethyl-phenyl)-propionyl]-piperidin-3-yl}-benzoic acid
Example 9-27: 4-{1-[3-(4-Isopropyl-phenyl)-propionyl]-piperidin-3-yl}-2-methyl-benzoic acid
Example 9-28: Isomer of 2-methoxy-5-{1-[4-methyl-2-(4-trifluoromethyl-phenyl)-thiazole-5-carbonyl]-piperidin-3-yl}-benzoic acid from L tartaric acid.
Example 9-29: Isomer of 2-methoxy-5-{1-[4-methyl-2-(4-trifluoromethyl-phenyl)-thiazole-5-carbonyl]-piperidin-3-yl}-benzoic acid from D tartaric acid
Example 9-30: 2-Fluoro-5-{1-[4-methyl-2-(4-trifluoromethyl-phenyl)-thiazole-5-carbonyl]-piperidin-3-yl}-benzoic acid
Example 9-3: 1 3-(3-Carboxy-4-fluoro-phenyl)-piperidine-1-carboxylic acid 4-trifluoromethyl-benzyl ester
Example 10: {3-[4-methyl-3-(1H-tetrazol-5-yl)-phenyl]-piperidin-1-yl}-[4-methyl-2-(4-trifluoromethyl-phenyl)-thiazol-5-yl]-methanone
Example 11: (S)-2-Methyl-2-(2-methyl-5-{1-[4-methyl-2-(4-trifluoromethyl-phenyl)-thiazole-5-carbonyl]-piperidin-3-yl}-phenoxyl)-propionic acid
Example 11-1: (S)-3-[3-(1-Carboxy-1-methyl-ethoxy)-4-methyl-phenyl]-piperidine-1-carboxylic acid 4-trifluoromethyl-benzyl ester
Example 11-2: (R)-2-Methyl-2-(2-methyl-5-{1-[4-methyl-2-(4-trifluoromethyl-phenyl)-thiazole-5-carbonyl]-piperidin-3-yl}-phenoxy)-propionic acid
Example 11-3: (R)-3-[3-(1-Carboxy-1-methyl-ethoxy)-4-methyl-phenyl]-piperidine-1-carboxylic acid 4-trifluoromethyl-benzyl ester
Examples 11-4, 11-5 and 11-6 were prepared using methods analogous to those described in Example 11 and 11-1.
Example 11-4: 2-Methyl-2-(2-methyl-4-{1-[4-methyl-2-(4-trifluoromethyl-phenyl)-thiazole-5-carbonyl]-piperidin-3-yl}-phenoxy)-propionic acid
Example 11-5: 3-[4-(1-Carboxy-1-methyl-ethoxy)-3-methyl-phenyl]-piperidine-1-carboxylic acid 4-trifluoromethyl-benzyl ester
Example 11-6: (S)-3-[3-(1-Carboxy-1-methyl-ethoxy)-4-methyl-phenyl]-piperidine-1-carboxylic acid 2-(4-trifluoromethyl-phenyl)-ethyl ester and (R)-3-[3-(1-Carboxy-1-methyl-ethoxy)-4-methyl-phenyl]-piperidine-1-carboxylic acid 2-(4-trifluoromethyl-phenyl)-ethyl ester
Example 12: (S)-(2-methyl-5-{1-[4-methyl-2-(4-trifluoromethyl-phenyl)-thiazole-5-carbonyl]-piperidin-3-yl}-phenoxy)-acetic acid.
Example 12-2: (R)-(2-Methyl-5-{1-[4-methyl-2-(4-trifluoromethyl-phenyl)-thiazole-5-carbonyl]-piperidin-3-yl}-phenoxy)-acetic acid
Example 12-3: (R)-3-(3-Carboxymethoxy-4-methyl-phenyl)-piperidine-1-carboxylic acid 4-trifluoromethyl-benzyl ester.
Example 12-4: (2-Methyl-4-{1-[4-methyl-2-(4-trifluoromethyl-phenyl)-thiazole-5-carbonyl]-piperidin-3-yl}-phenoxy)-acetic acid
Example 12-5: 3-(4-Carboxymethoxy-3-methyl-phenyl)-piperidine-1-carboxylic acid 4-trifluoromethyl-benzyl ester
Example 13: C,C,C-Trifluoro-N-(2-methyl-5-{1-[4-methyl-2-(4-trifluoromethyl-phenyl)-thiazole-5-carbonyl]-piperidin-3-yl}-phenyl)-methanesulfonamide
Example 13-1: [3-(Carboxymethyl-amino)-4-methyl-phenyl]-piperidine-1-carboxylic acid 4-trifluoromethyl-benzyl ester
Example 13-2: (2-Methyl-5-{1-[4-methyl-2-(4-trifluoromethyl-phenyl)-thiazole-5-carbonyl]-piperidin-3-yl}-phenylamino)-acetic acid

1. A method of palliative, prophylactic or curative treatment of negative energy balance in a ruminant, comprising administering to the ruminant an effective amount of a compound of formula I: 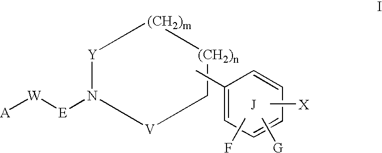
an isomer thereof, a prodrug of said compound or isomer, or a pharmaceutically acceptable salt of said compound, isomer or prodrug;
wherein m and n are each independently one or two;
V and Y are each independently a) methylene, or b) carbonyl;
F and G are each independently a) hydrogen, b) halo, c) (C₁-C₄)alkyl optionally substituted with one to nine fluoro, d) (C₃-C₆)cycloalkyl, e) hydroxy, f) (C₁-C₄)alkoxy or g) (C₁-C₄)alkylthio;
X is a) —Z or b) —B—C(R¹R²)—Z;
B is a) oxy, b) thio, c) sulfinyl, d) sulfonyl, e) methylene, or f) —N(H)—;
Z is a) —C(O)OH, b) —C(O)O-(C₁-C₄)alkyl, c) —C(O)O-(C₀-C₄)alkyl-aryl, d) —C(O)—NH₂, e) hydroxyaminocarbonyl, f) tetrazolyl, g) tetrazolylaminocarbonyl, h) 4,5-dihydro-5-oxo-1,2,4-oxadiazol-3-yl, i) 3-oxoisoxazolidin-4-yl-aminocarbonyl, j) —C(O)N(H)SO₂R⁴, or k) —NHSO₂R⁴; wherein R⁴ is a) (C₁-C₆)alkyl, b) amino or c) mono-N- or di-N,N-(C₁-C₆)alkylamino, wherein the (C₁-C₆)alkyl substituents in R⁴ are optionally independently substituted with one to nine fluoro;
R¹ is a) H, b) (C₁-C₄)alkyl, or c) (C₃-C₆)cycloalkyl;
R is a) H, b) (C₃-C₆)cycloalkyl or c) a fully or partially saturated or fully unsaturated one to four membered straight or branched carbon chain; wherein the carbon(s) in the carbon chain may optionally be replaced with one or two heteroatoms selected independently from oxygen and sulfur; and wherein the sulfur is optionally mono- or di-substituted with oxo;
wherein the carbon(s) in the carbon chain in R² is optionally independently substituted as follows: a) the carbon(s) is optionally mono-, di- or tri-substituted independently with halo, b) the carbon(s) is optionally mono-substituted with hydroxy or (C₁-C₄)alkoxy, and c) the carbon(s) is optionally mono-substituted with oxo; and
wherein the carbon(s) in the carbon chain in R² is optionally mono-substituted with Q;
wherein Q is a partially or fully saturated or fully unsaturated three to eight membered ring optionally having one to four heteroatoms selected independently from oxygen, sulfur and nitrogen, or is a bicyclic ring consisting of two fused partially or fully saturated or fully unsaturated three to six membered rings, taken independently; wherein the bicyclic ring optionally has one to four heteroatoms selected independently from oxygen, sulfur and nitrogen;
wherein the Q ring is optionally mono-, di- or tri-substituted independently with a) halo, b) (C₂-C₆)alkenyl, c) (C₁-C₆) alkyl, d) hydroxy, e) (C₁-C₆)alkoxy, f) (C₁-C₄)alkylthio, g) amino, h) nitro, i) cyano, j) oxo, k) carboxy, l) (C₁-C₆)alkyloxycarbonyl, or m) mono-N- or di-N,N-(C₁-C₆)alkylamino; wherein the (C₁-C₆)alkyl and (C₁-C₆)alkoxy substituents on the Q ring is optionally mono-, di- or tri-substituted independently with a) halo, b) hydroxy, c) (C₁-C₆)alkoxy, d) (C₁-C₄)alkylthio, e) amino, f) nitro, g) cyano, h) oxo, i) carboxy, j) (C₁-C₆)alkyloxycarbonyl, or k) mono-N- or di-N,N-(C₁-C₆)alkylamino; wherein the (C₁-C₆)alkyl substituent is on the Q ring is also optionally substituted with one to nine fluoro;
or wherein R¹ and R² are linked together to form a three to six membered fully saturated carbocyclic ring, optionally having one heteroatom selected from oxygen, sulfur and nitrogen to form a heterocyclic ring;
E is a) carbonyl, b) sulfonyl, or c) methylene;
W is a) a bond, b) carbonyl, c) —N(H)—, d) —N((C₁-C₄)alkyl)-, e) (C₂-C₈)alkenyl, f) oxy, g) -(C₁-C₄)alkyl-O-, h) —NH-(C₁-C₄)alkyl-, or i) -(C₁-C₆)alkyl-; wherein the (C₁-C₆)alkyl and the (C₂-C₈)alkenyl groups in W may optionally be mono- or di-substituted independently with a) oxo, b) halo, c) (C₁-C₆)alkoxycarbonyl, d) (C₁-C₆)alkyl, e) (C₂-C₆)alkenyl, f) (C₃-C₇)cycloalkyl, g) hydroxy, h) (C₁-C₆)alkoxy, i) (C₁-C₄)alkylthio, j) amino, k) cyano, l) nitro, m) mono-N- or di-N,N-(C₁-C₆)alkylamino, or n) —NH-(C₁-C)alkylamino;
or wherein W is CR⁷R⁸ wherein R⁷ and R⁸ are linked together to form a three to six membered fully saturated carbocyclic ring;
A is a) mono-N- or di-N,N-(C₁-C₆)alkylamino, b) (C₂-C₆)alkanoylamino, c) (C₁-C₆)alkoxy, d) a partially or fully saturated or fully unsaturated three to eight membered ring optionally having one to four heteroatoms selected independently from oxygen, sulfur and nitrogen, or e) a bicyclic ring consisting of two fused partially or fully saturated or fully unsaturated three to six membered rings, taken independently; wherein the bicyclic ring optionally has one to four heteroatoms selected independently from oxygen, sulfur and nitrogen; and
wherein the A ring is optionally mono-, di- or tri-substituted independently with a) oxo, b) carboxy, c) halo, d) (C₁-C₆)alkoxycarbonyl, e) (C₁-C₆)alkyl, f) (C₂-C₆)alkenyl, g) (C₃-C₇)cycloalkyl, h) (C₃-C₇)cycloalkyl(C₁-C₆)alkyl, i) hydroxy, j) (C₁-C₆)alkoxy, k) (C₁-C₄)alkylthio, l) (C₁-C₄)alkylsulfonyl, m) amino, n) cyano, o) nitro, or p) mono-N- or di-N,N-(C₁-C₆)alkylamino; wherein the (C₁-C₆)alkyl and (C₁-C₆)alkoxy substituents on the A ring are also optionally mono-, di- or tri-substituted independently with a) halo, b) hydroxy, c) (C₁-C₄)alkyl optionally substituted with one to nine fluoro, d) (C₃-C₆)cycloalkyl, e) (C₁-C₆)alkoxy, f) amino, or g) mono-N- or di-N,N-(C₁-C₆)alkylamino;
or wherein the A ring is optionally mono-substituted with a partially or fully saturated or fully unsaturated three to eight membered ring, optionally having one to four heteroatoms selected independently from oxygen, sulfur and nitrogen; also wherein this three to eight membered ring is optionally mono-, di- or tri-substituted independently with a) halo, b) hydroxy, c) (C₁-C₄)alkyl optionally substituted with one to nine fluoro, d) (C₃-C₇)cycloalkyl, e) (C₁-C₆)alkoxy optionally substituted with one to nine fluoro, f) amino, g) mono-N- or di-N,N-(C₁-C₆)alkylamino, or h) (C₁-C₄)alkylthio;
provided that:
1) when V and Y are each methylene and m and n are each one forming a six-membered piperidinyl ring, this ring is substituted by the phenyl ring (designated as J) at other than the 4-position;
2) when E is carbonyl, W is a bond and X is —B—C(R¹R²)—Z wherein R¹ and R² are each hydrogen, B is —O— or —N(H)—, and Z is —C(O)OH or —C(O)O-(C₁-C₄)alkyl, then one of F or G must be a) -(C₁-C₄)alkyl, b) (C₃-C₆)cycloalkyl, c) (C₁-C₄)alkoxy or d) (C₁-C₄)alkylthio.

2. The method according to claim 1, further provided that: when E is carbonyl, W is a bond, X is —Z, and Z is —C(O)OH, —C(O)O-(C₁-C₄)alkyl, —C(O)NH₂, then one of F or G must be a) -(C₁-C₄)alkyl, b) (C₃-C₆)cycloalkyl, c) (C₁-C₄)alkoxy or d) (C₁-C₄)alkylthio.

3. The method according to claim 1, wherein:
V and Y are each methylene or one of V and Y is carbonyl and the other is methylene;
E is carbonyl;
W is a) a bond, b) oxy, c) —N(H)—, d) —N(H)-(C₁-C₄)alkyl-, e) -(C₁-C₄)alkyl-, f) -(C₁-C₄)alkyl-O- or g) —CR⁷R⁸— wherein R⁷ and R⁸ are linked together to form a three-membered fully saturated carbocyclic ring; and
A is a partially or fully saturated or fully unsaturated three to eight membered ring optionally having one to four heteroatoms selected independently from oxygen, sulfur and nitrogen;
wherein the A ring is optionally mono-, di- or tri-substituted independently with a) oxo, b) carboxy, c) halo, d) (C₁-C₆)alkoxycarbonyl, e) (C₁-C₆)alkyl, f) (C₂-C₆)alkenyl, g) (C₃-C₇)cycloalkyl, h) (C₃-C₇)cycloalkyl(C₁-C₆)alkyl, i) hydroxy, j) (C₁-C₆)alkoxy, k) (C₁-C₄)alkylthio, l) (C₁-C₄)alkylsulfonyl, m) amino, n) cyano, o) nitro, or p) mono-N- or di-N,N-(C₁-C₆)alkylamino; wherein the (C₁-C₆)alkyl and (C₁-C₆)alkoxy substituents on the A ring are also optionally mono-, di- or tri-substituted independently with a) halo, b) hydroxy, c) (C₁-C₄)alkyl optionally substituted with one to nine fluoro, d) (C₃-C₆)cycloalkyl, e) (C₁-C₆)alkoxy, f) amino, or g) mono-N- or di-N,N-(C₁-C₆)alkylamino;
or wherein the A ring is optionally mono-substituted with a partially or fully saturated or fully unsaturated three to eight membered ring, optionally having one to four heteroatoms selected independently from oxygen, sulfur and nitrogen; also wherein this three to eight membered ring is optionally mono-, di- or tri-substituted independently with a) halo, b) hydroxy, c) (C₁-C₆)alkyl optionally substituted with one to nine fluoro, d) (C₃-C₇)cycloalkyl, e) (C₁-C₆)alkoxy optionally substituted with one to nine fluoro, f) amino, g) mono-N- or di-N,N-(C₁-C₆)alkylamino, or h) (C₁-C₄)alkylthio;

4. The method according to claims 1 or 3, wherein:
A is a) phenyl optionally independently substituted with one or two 1) -(C₁-C₆)alkyl, 2) —CF₃, 3) —OCF₃ 4) -(C₁-C₆)alkoxy, 5) (C₃-C₇)cycloalkyl, 6) halo 7) -(C₁-C₄)alkylthio or 8) hydroxy; or b) thiazolyl optionally independently substituted with 1) one or two methyl or 2) phenyl optionally independently substituted with one or two a) -(C₁-C₆)alkyl, b) —CF₃, c) —OCF₃, d) -(C₁-C₆)alkoxy, e) (C₃-C₇)cycloalkyl, f) halo, g) -(C₁-C₄)alkylthio or h) hydroxy;
F and G are each independently a) hydrogen, b) halo, c) (C₁-C₄)alkyl or d) (C₁-C₄)alkoxy;
X is a) —Z or b) —B—C(R¹R²)—Z;
B is a) oxy, b) thio or c) —N(H)—;
Z is a) —C(O)OH, b) —C(O)O-(C₁-C₄)alkyl, c) —C(O)NH₂ or d) tetrazolyl;
R¹ is a) hydrogen or b) methyl; and
R² is a) hydrogen or b) a fully or partially saturated or fully unsaturated one to four membered straight or branched carbon chain; wherein the carbon(s) in the carbon chain may optionally be replaced with one or two heteroatoms selected independently from oxygen and sulfur;
wherein the carbon(s) in the carbon chain in R² is optionally mono-substituted with Q;
wherein Q is a partially or fully saturated or fully unsaturated three to eight membered ring optionally having one to four heteroatoms selected independently from oxygen, sulfur and nitrogen.

5. The method according to claim 4, wherein:
R¹ is a) hydrogen or b) methyl;
R² is a) hydrogen, b) methyl or c) —O—CH₂-phenyl;
m is one, n is one and V and Y are each methylene to form a piperdinyl ring;
X is —B—C(R¹R²)—Z;
B is oxy; and
the phenyl ring (designated as J) is attached at the 3-position of the piperidinyl ring.

6. The method according to claim 5, wherein the compound of formula I is of formula I-A or formula I-C: 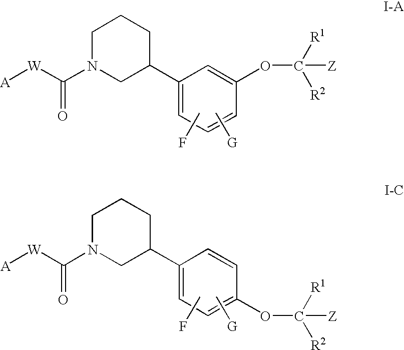
wherein R¹ and R² are each independently a) hydrogen or b) methyl;
F and G are each independently a) hydrogen or b) methyl; and
Z is —C(O)OH.

7. The method according to claim 6 wherein:
W is a) oxy, b) —N(H)—, c) —N(H)-(C₁-C₄)alkyl-, d) -(C₁-C₄)alkyl- or e) -(C₁-C₄)alkyl-O-; and A is phenyl optionally substituted with a) -(C₁-C₄)alkyl, b) —CF₃, c) —OCF₃ d) -(C₁-C₄)alkoxy, e) cyclopropyl, f) halo, g) -(C₁-C₄)alkylthio or h) hydroxy; or
W is a bond; and A is thiazolyl optionally substituted with a) one or two -methyl, or b) -phenyl optionally substituted with 1) -(C₁-C₄)alkyl, 2) —CF₃, 3) —OCF₃ 4) -C₁-C₄)alkoxy, 5) cyclopropyl, 6) halo or 7) -(C₁-C₄)alkylthio.

8. The method according to claim 4, wherein:
m is one, n is one and V and Y are each methylene to form a piperidinyl ring;
X is —Z;
and
the phenyl ring (designated as J) is attached at the 3-position of the piperidinyl ring.

9. The method according to claim 8 wherein the compound of formula I is a compound of the formula I-B or formula I-D 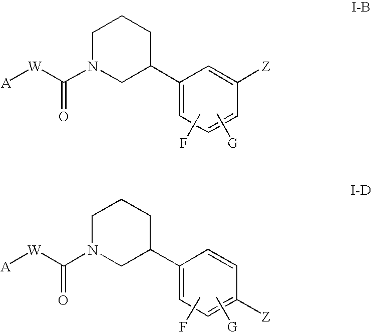
wherein F and G are each a) hydrogen, b) methyl, c) fluoro or d) methoxy; and
Z is a) —C(O)OH, b) —C(O)O-(C₁-C₄)alkyl or c) —C(O)NH₂.

10. The method according to claim 9 wherein
W is a) -(C₁-C₄)alkyl- or b) -(C₁-C₄)alkyl-O-; and A is phenyl optionally substituted with a) -(C₁-C₄)alkyl, b) —CF₃, c) —OCF₃, d) -(C₁-C₄)alkoxy, e) cyclopropyl, f) halo, g) -(C₁-C₄)alkylthio, or h) hydroxy; or
W is a bond; and A is a) thiazolyl optionally substituted with 1) one or two -methyl or 2) -phenyl optionally substituted with i) -(C₁-C₄)alkyl, ii) —CF₃, iii) —OCF₃ iv) -(C₁-C₄)alkoxy, v) cyclopropyl or vi) halo; or b) phenyl optionally substituted with 1) -(C₁-C₄)alkyl, 2) —CF₃, 3) —OCF₃ 4) -(C₁-C₄)alkoxy, 5) cyclopropyl, 6) halo, or 7) -(C₁-C₄)alkylthio.

11. The method according to claim 1 wherein the compound of formula I is selected from:
2-{3-[1-(4-Isopropyl-phenylcarbamoyl)-piperidin-3-yl]-phenoxy}-2-methyl-propionic acid;
(S)-2-{3-[1-(4-Isopropyl-phenylcarbamoyl)-piperidin-3-yl]-phenoxy}-2-methyl-propionic acid;
(R)-2-{3-[1-(4-Isopropyl-phenylcarbamoyl)-piperidin-3-yl]-phenoxy}-2-methyl-propionic acid;
2-Methyl-2-(3-{1-[(4-trifluoromethyl-phenyl)-acetyl]-piperidin-3-yl}-phenoxy)-propionic acid;
(S)-2-Methyl-2-(3-{1-[(4-trifluoromethyl-phenyl)-acetyl]-piperidin-3-yl}-phenoxy)-propionic acid;
(R)-2-Methyl-2-(3-{1-[(4-trifluoromethyl-phenyl)-acetyl]-piperidin-3-yl}-phenoxy)-propionic acid;
2-(3-{1-[(4-Isopropyl-phenyl)-acetyl]-piperidin-3-yl}-phenoxy)-2-methyl-propionic acid;
(S)-2-(3-{1-[(4-Isopropyl-phenyl)-acetyl]-piperidin-3-yl}-phenoxy)-2-methyl-propionic acid;
(R)-2-(3-{1-[(4-Isopropyl-phenyl)-acetyl]-piperidin-3-yl}-phenoxy)-2-methyl-propionic acid;
2-(3-{1-[3-(4-Isopropyl-phenyl)-propionyl]-piperidin-3-yl}-phenoxy)-2-methyl-propionic acid;
(S)-2-(3-{1-[3-(4-Isopropyl-phenyl)-propionyl]-piperidin-3-yl}-phenoxy)-2-methyl-propionic acid;
(R)-2-(3-{1-[3-(4-Isopropyl-phenyl)-propionyl]-piperidin-3-yl}-phenoxy)-2-methyl-propionic acid;
2-(3-{1-[(4-Isopropyl-phenoxy)-acetyl]-piperidin-3-yl}-phenoxy)-2-methyl-propionic acid;
(S)-2-(3-{1-[(4-Isopropyl-phenoxy)-acetyl] -piperidin-3-yl}-phenoxy)-2-methyl-propionic acid;
(R)-2-(3-{1-[(4-Isopropyl-phenoxy)-acetyl]-piperidin-3-yl}-phenoxy)-2-methyl-propionic acid;
2-(3-{1-[2-(4-Isopropyl-phenoxy)-2-methyl-propionyl]-piperidin-3-yl}-phenoxy)-2-methyl-propionic acid;
(S)-2-(3-{1-[2-(4-Isopropyl-phenoxy)-2-methyl-propionyl]-piperidin-3-yl}-phenoxy)-2-methyl-propionic acid;
(R)-2-(3-{1-[2-(4-Isopropyl-phenoxy)-2-methyl-propionyl]-piperidin-3-yl}-phenoxy)-2-methyl-propionic acid;
2-Methyl-2-(3-{1-[3-(4-trifluoromethyl-phenyl)-propionyl]-piperidin-3-yl}-phenoxy)-propionic acid;
(S)-2-Methyl-2-(3-{1-[3-(4-trifluoromethyl-phenyl)-propionyl]-piperidin-3-yl}-phenoxy)-propionic acid;
(R)-2-Methyl-2-(3-{1-[3-(4-trifluoromethyl-phenyl)-propionyl]-piperidin-3-yl}-phenoxy)-propionic acid;
2-Methyl-2-(3-{1-[(4-trifluoromethoxy-phenoxy)-acetyl]-piperidin-3-yl}-phenoxy)-propionic acid;
(S)-2-Methyl-2-(3-{1-[(4-trifluoromethoxy-phenoxy)-acetyl]-piperidin-3-yl}-phenoxy)-propionic acid;
(R)-2-Methyl-2-(3-{1 [(4-trifluoromethoxy-phenoxy)-acetyl]-piperidin-3-yl}-phenoxy)-propionic acid;
(3-{1-[(4-Isopropyl-phenyl)-acetyl]-piperidin-3-yl}-phenoxy)-acetic acid;
(S)-(3-{1-[(4-Isopropyl-phenyl)-acetyl]-piperidin-3-yl}-phenoxy)-acetic acid;
(R)-(3-{1-[(4-Isopropyl-phenyl)-acetyl]-piperidin-3-yl}-phenoxy)-acetic acid;
3-[3-(1-Carboxy-1-methyl-ethoxy)-phenyl]-piperidine-1-carboxylic acid 4-isopropyl-phenyl ester;
(S)-3-[3-(1-Carboxy-1-methyl-ethoxy)-phenyl]-piperidine-1-carboxylic acid 4-isopropyl-phenyl ester;
(R)-3-[3-(1-Carboxy-1-methyl-ethoxy)-phenyl]-piperidine-1-carboxylic acid 4-isopropyl-phenyl ester;
(S)-2-(3-{1-[(4-tert-Butyl-phenyl)-acetyl]-piperidin-3-yl}-phenoxy)-2-methyl-propionic acid;
(R)-2-(3-{1-[(4-tert-Butyl-phenyl)-acetyl]-piperidin-3-yl}-phenoxy)-2-methyl-propionic acid;
2-(3-{1-[(4-tert-Butyl-phenyl)-acetyl]-piperidin-3-yl}-phenoxy)-2-methyl-propionic acid;
(S)-2-Methyl-2-(3-{1-[(4-trifluoromethoxy-phenyl)-acetyl]-piperidin-3-yl}-phenoxy)-propionic acid;
(R)-2-Methyl-2-(3-{1-[(4-trifluoromethoxy-phenyl)-acetyl]-piperidin-3-yl}-phenoxy)-propionic acid;
2-Methyl-2-(3-{1-[(4-trifluoromethoxy-phenyl)-acetyl]-piperidin-3-yl}-phenoxy)-propionic acid;
(S)-3-[3-(1-Carboxy-1-methyl-ethoxy)-phenyl]-piperidine-1-carboxylic acid 4-isopropyl-benzyl ester;
(R)-3-[3-(1-Carboxy-1-methyl-ethoxy)-phenyl]-piperidine-1-carboxylic acid 4-isopropyl-benzyl ester;
3-[3-(1-Carboxy-1-methyl-ethoxy)-phenyl]-piperidine-1-carboxylic acid 4-isopropyl-benzyl ester;
(S)-3-[3-(1-Carboxy-1-methyl-ethoxy)-phenyl]-piperidine-1-carboxylic acid 4-isopropyl-phenyl ester;
(R)-3-[3-(1-Carboxy-1-methyl-ethoxy)-phenyl]-piperidine-1-carboxylic acid 4-isopropyl-phenyl ester;
3-[3-(1-Carboxy-1-methyl-ethoxy)-phenyl]-piperidine-1-carboxylic acid 4-isopropyl-phenyl ester;
2-{3-[1-(4-Isopropyl-benzylcarbamoyl)-piperidin-3-yl]-phenoxy}-2-methyl-propionic acid;
(S)-2-{3-[1-(4-Isopropyl-benzylcarbamoyl)-piperidin-3-yl]-phenoxy}-2-methyl-propionic acid;
(R)-2-{3-[1-(4-Isopropyl-benzylcarbamoyl)-piperidin-3-yl]-phenoxy}-2-methyl-propionic acid;
3-[3-(1-Carboxy-1-methyl-ethoxy)-phenyl]-piperidine-1-carboxylic acid 4-trifluoromethyl-benzyl ester;
(S)-3-[3-(1-Carboxy-1-methyl-ethoxy)-phenyl]-piperidine-1-carboxylic acid 4-trifluoromethyl-benzyl ester;
(R)-3-[3-(1-Carboxy-1-methyl-ethoxy)-phenyl]-piperidine-1-carboxylic acid 4-trifluoromethyl-benzyl ester;
(S)-2-Methyl-2-{3-[1-(4-trifluoromethoxy-benzylcarbamoyl)-piperidin-3-yl]-phenoxy}-propionic acid;
(R)-2-Methyl-2-{3-[1-(4-trifluoromethoxy-benzylcarbamoyl)-piperidin-3-yl]-phenoxy}-propionic acid;
2-Methyl-2-{3-[1-(4-trifluoromethoxy-benzylcarbamoyl)-piperidin-3-yl]-phenoxy}-propionic acid;
3-[3-(1-Carboxy-1-methyl-ethoxy)-phenyl]-piperidine-1-carboxylic acid 4-cyclopropyl-benzyl ester;
(S)-3-[3-(1-Carboxy-1-methyl-ethoxy)-phenyl]-piperidine-1-carboxylic acid 4-cyclopropyl-benzyl ester;
(R)-3-[3-(1-Carboxy-1-methyl-ethoxy)-phenyl]-piperidine-1-carboxylic acid 4-cyclopropyl-benzyl ester;
(S)-3-(3-carboxymethoxy-4-methyl-phenyl)-piperidine-1-carboxylic acid 4-trifluoromethyl-benzyl ester;
(R)-3-(3-carboxymethoxy-4-methyl-phenyl)-piperidine-1-carboxylic acid 4-trifluoromethyl-benzyl ester;
3-(3-carboxymethoxy-4-methyl-phenyl)-piperidine-1-carboxylic acid 4-trifluoromethyl-benzyl ester;
(S)-3-[3-(1-Carboxy-1-methyl-ethoxy)-4-methyl-phenyl]-piperidine-1-carboxylic acid 4-trifluoromethyl-benzyl ester;
(R)-3-[3-(1-Carboxy-1-methyl-ethoxy)-4-methyl-phenyl]-piperidine-1-carboxylic acid 4-trifluoromethyl-benzyl ester;
3-[3-(1-Carboxy-1-methyl-ethoxy)-4-methyl-phenyl]-piperidine-1-carboxylic acid 4-trifluoromethyl-benzyl ester;
(S)-2-Methyl-2-(3-{1-[4-methyl-2-(4-trifluoromethyl-phenyl)-thiazole-5-carbonyl]-piperidin-3-yl}-phenoxy)-propionic acid;
(R)-2-Methyl-2-(3-{1-[4-methyl-2-(4-trifluoromethyl-phenyl)-thiazole-5-carbonyl]-piperidin-3-yl}-phenoxy)-propionic acid;
2-Methyl-2-(3-{1-[4-methyl-2-(4-trifluoromethyl-phenyl)-thiazole-5-carbonyl]-piperidin-3-yl}-phenoxy)-propionic acid;
(S)-(2-methyl-5-{1-[4-methyl-2-(4-trifluoromethyl-phenyl)-thiazole-5-carbonyl]-piperidin-3-yl}-phenoxy)-acetic acid;
(R)-(2-methyl-5-{1-[4-methyl-2-(4-trifluoromethyl-phenyl)-thiazole-5-carbonyl]-piperidin-3-yl}-phenoxy)-acetic acid;
(2-methyl-5-{1-[4-methyl-2-(4-trifluoromethyl-phenyl)-thiazole-5-carbonyl]-piperidin-3-yl}-phenoxy)-acetic acid;
(S)-2-Methyl-2-(2-methyl-5-{1-[4-methyl-2-(4-trifluoromethyl-phenyl)-thiazole-5-carbonyl]-piperidin-3-yl}-phenoxy)-propionic acid;
(R)-2-Methyl-2-(2-methyl-5-{1-[4-methyl-2-(4-trifluoromethyl-phenyl)-thiazole-5-carbonyl]-piperidin-3-yl}-phenoxy)-propionic acid;
2-Methyl-2-(2-methyl-5-{1-[4-methyl-2-(4-trifluoromethyl-phenyl)-thiazole-5-carbonyl]-piperidin-3-yl}-phenoxy)-propionic acid;
(R)-3-[3-(1-Carboxy-1-methyl-ethoxy)-phenyl]-piperidine-1-carboxylic acid 3-trifluoromethyl-benzyl ester;
(S)-3-[3-(1-Carboxy-1-methyl-ethoxy)-phenyl]-piperidine-1-carboxylic acid 3-trifluoromethyl-benzyl ester;
3-[3-(1-Carboxy-1-methyl-ethoxy)-phenyl]-piperidine-1-carboxylic acid 3-trifluoromethyl-benzyl ester;
(S)-3-[3-(1-Carboxy-1-methyl-ethoxy)-4-methyl-phenyl]-piperidine-1-carboxylic acid 2-(4-trifluoromethyl-phenyl)-ethyl ester;
(R)-3-[3-(1-Carboxy-1-methyl-ethoxy)-4-methyl-phenyl]-piperidine-1-carboxylic acid 2-(4-trifluoromethyl-phenyl)-ethyl ester; and
3-[3-(1-Carboxy-1-methyl-ethoxy)-4-methyl-phenyl]-piperidine-1-carboxylic acid 2-(4-trifluoromethyl-phenyl)-ethyl ester.

12. A method for the palliative, prophylactic or curative treatment of ruminant disease associated with negative energy balance in a ruminant, comprising administering to the ruminant an effective amount of a compound of formula I: 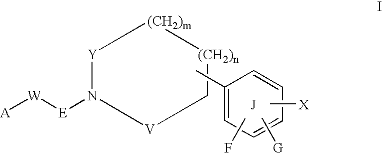
an isomer thereof, a prodrug of said compound or isomer, or a pharmaceutically acceptable salt of said compound, isomer or prodrug;
wherein m and n are each independently one or two;
V and Y are each independently a) methylene, or b) carbonyl;
F and G are each independently a) hydrogen, b) halo, c) (C₁-C₄)alkyl optionally substituted with one to nine fluoro, d) (C₃-C₆)cycloalkyl, e) hydroxy, f) (C₁-C₄ )alkoxy or g) (C₁-C₄alkylthio;
X is a) —Z or b) —B—C(R¹R²)—Z;
B is a) oxy, b) thio, c) sulfinyl, d) sulfonyl, e) methylene, or f) —N(H)—;
Z is a) —C(O)OH, b) —C(O)O-(C₁-C₄)alkyl, c) —C(O)O-(C₀-C₄)alkyl-aryl, d) —C(O)—NH₂, e) hydroxyaminocarbonyl, f) tetrazolyl, g) tetrazolylaminocarbonyl, h) 4,5-dihydro-5-oxo-1,2,4-oxadiazol-3-yl, i) 3-oxoisoxazolidin-4-yl-aminocarbonyl j) —C(O)N(H)SO₂R⁴, or k) —NHSO₂R⁴; wherein R⁴ is a) (C₁-C₆)alkyl, b) amino or c) mono-N- or di-N,N-(C₁-C₆)alkylamino, wherein the (C₁-C₆)alkyl substituents in R⁴ are optionally independently substituted with one to nine fluoro:
R¹ is a) H, b) (C₁-C₄)alkyl, or c) (C₃-C₆)cycloalkyl;
R² is a) H, b) (C₃-C₆)cycloalkyl or c) a fully or partially saturated or fully unsaturated one to four membered straight or branched carbon chain; wherein the carbon(s) in the carbon chain may optionally be replaced with one or two heteroatoms selected independently from oxygen and sulfur; and wherein the sulfur is optionally mono- or di-substituted with oxo;
wherein the carbon(s) in the carbon chain in R² is optionally independently substituted as follows: a) the carbon(s) is optionally mono-, di- or tri-substituted independently with halo, b) the carbon(s) is optionally mono-substituted with hydroxy or (C₁-C₄)alkoxy, and c) the carbon(s) is optionally mono-substituted with oxo; and
wherein the carbon(s) in the carbon chain in R² is optionally mono-substituted with Q;
wherein Q is a partially or fully saturated or fully unsaturated three to eight membered ring optionally having one to four heteroatoms selected independently from oxygen, sulfur and nitrogen, or is a bicyclic ring consisting of two fused partially or fully saturated or fully unsaturated three to six membered rings, taken independently; wherein the bicyclic ring optionally has one to four heteroatoms selected independently from oxygen, sulfur and nitrogen;
wherein the Q ring is optionally mono-, di- or tri-substituted independently with a) halo, b) (C₂-C₆)alkenyl, c) (C₁-C₆) alkyl, d) hydroxy, e) (C₁-C₆)alkoxy, f) (C₁-C₄)alkylthio, g) amino, h) nitro, i) cyano, j) oxo, k) carboxy, l) (C₁-C₆)alkyloxycarbonyl, or m) mono-N- or di-N,N-(C₁-C₆)alkylamino; wherein the (C₁-C₆)alkyl and (C₁-C₆)alkoxy substituents on the Q ring is optionally mono-, di- or tri-substituted independently with a) halo, b) hydroxy, c) (C₁-C₆)alkoxy, d) (C₁-C₄)alkylthio, e) amino, f) nitro, g) cyano, h) oxo, i) carboxy, j) (C₁-C₆)alkyloxycarbonyl, or k) mono-N- or di-N,N-(C₁-C₆)alkylamino; wherein the (C₁-C₆)alkyl substituent is on the Q ring is also optionally substituted with one to nine fluoro;
or wherein R¹ and R² are linked together to form a three to six membered fully saturated carbocyclic ring, optionally having one heteroatom selected from oxygen, sulfur and nitrogen to form a heterocyclic ring;
E is a) carbonyl, b) sulfonyl, or c) methylene;
W is a) a bond, b) carbonyl, c) —N(H)—, d) —N((C₁-C₄)alkyl)-, e) (C₂-C₈)alkenyl, f) oxy, g) -(C₁-C₄)alkyl-O-, h) —NH-(C₁-C₄)alkyl-, or i) -(C₁-C₆)alkyl-; wherein the (C₁-C₆)alkyl and the (C₂-C₈)alkenyl groups in W may optionally be mono- or di-substituted independently with a) oxo, b) halo, c) (C₁-C₆)alkoxycarbonyl, d) (C₁-C₆)alkyl, e) (C₂-C₆)alkenyl, f) (C₃-C₇)cycloalkyl, g) hydroxy, h) (C₁-C₆)alkoxy, i) (C₁-C₄)alkylthio, j) amino, k) cyano, l) nitro, m) mono-N- or di-N,N-(C₁-C₆)alkylamino, or n) —NH-(C₁-C)alkylamino;
or wherein W is CR⁷R⁸ wherein R⁷ and R⁸ are linked together to form a three to six membered fully saturated carbocyclic ring;
A is a) mono-N- or di-N,N-(C₁-C₆)alkylamino, b) (C₂-C₆)alkanoylamino, c) (C₁-C₆)alkoxy, d) a partially or fully saturated or fully unsaturated three to eight membered ring optionally having one to four heteroatoms selected independently from oxygen, sulfur and nitrogen, or e) a bicyclic ring consisting of two fused partially or fully saturated or fully unsaturated three to six membered rings, taken independently; wherein the bicyclic ring optionally has one to four heteroatoms selected independently from oxygen, sulfur and nitrogen; and
wherein the A ring is optionally mono-, di- or tri-substituted independently with a) oxo, b) carboxy, c) halo, d) (C₁-C₆)alkoxycarbonyl, e) (C₁-C₆)alkyl, f) (C₂-C₆)alkenyl, g) (C₃-C₇)cycloalkyl, h) (C₃-C₇)cycloalkyl(C₁-C₆)alkyl, i) hydroxy, j) (C₁-C₆)alkoxy, k) (C₁-C₄)alkylthio, l) (C₁-C₄)alkylsulfonyl, m) amino, n) cyano, o) nitro, or p) mono-N- or di-N,N-(C₁-C₆)alkylamino; wherein the (C₁-C₆)alkyl and (C₁-C₆)alkoxy substituents on the A ring are also optionally mono-, di- or tri-substituted independently with a) halo, b) hydroxy, c) (C₁-C₄)alkyl optionally substituted with one to nine fluoro, d) (C₃-C₆)cycloalkyl, e) (C₁-C₆)alkoxy, f) amino, or g) mono-N- or di-N,N-(C₁-C₆)alkylamino;
or wherein the A ring is optionally mono-substituted with a partially or fully saturated or fully unsaturated three to eight membered ring, optionally having one to four heteroatoms selected independently from oxygen, sulfur and nitrogen; also wherein this three to eight membered ring is optionally mono-, di- or tri-substituted independently with a) halo, b) hydroxy, c) (C₁-C₄)alkyl optionally substituted with one to nine fluoro, d) (C₃-C₇)cycloalkyl, e) (C₁-C₆)alkoxy optionally substituted with one to nine fluoro, f) amino, g) mono-N- or di-N,N-(C₁-C₆)alkylamino, or h) (C₁-C₄)alkylthio;
provided that:
1) when V and Y are each methylene and m and n are each one forming a six-membered piperidinyl ring, this ring is substituted by the phenyl ring (designated as J) at other than the 4-position;
2) when E is carbonyl, W is a bond and X is —B—C(R¹R²)—Z wherein R¹ and R² are each hydrogen, B is —O— or —N(H)—, and Z is —C(O)OH or —C(O)O-(C₁-C₄)alkyl, then one of F or G must be a) -(C₁-C₄)alkyl, b) (C₃-C₆)cycloalkyl, c) (C₁-C₄)alkoxy or d) (C₁-C₄)alkylthio.

13. The method according to claim 12 wherein the ruminant disease associated with negative energy balance in ruminants is selected from fatty liver syndrome, dystocia, immune dysfunction, impaired immune function, toxification, primary ketosis, secondary ketosis, downer cow syndrome, indigestion, inappetence, retained placenta, displaced abomasum, mastitis, (endo-)-metritis, infertility, low fertility, lameness, subacute rumen acidosis and inadequate nutrient intake associated with stress.

14. The method according to claim 1 or 13 wherein the compound of formula I is administered during the period from 30 days prepartum to 70 days postpartum.

15. The method according to claim 14 wherein the compound of formula I is administered up to three times during the first seven days postpartum.

16. The method according to claim 15 wherein the compound of formula I is administered once during the first 24 hours postpartum.

17. A method of increasing ruminant milk quality or yield comprising administering to a ruminant an effective amount of a compound of formula I: 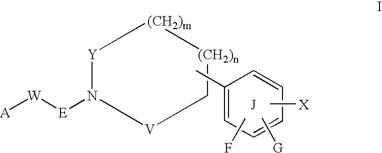
an isomer thereof, a prodrug of said compound or isomer, or a pharmaceutically acceptable salt of said compound, isomer or prodrug;
wherein m and n are each independently one or two;
V and Y are each independently a) methylene, or b) carbonyl:
F and G are each independently a) hydrogen, b) halo, c) (C₁-C₄)alkyl optionally substituted with one to nine fluoro, d) (C₃-C₆)cycloalkyl, e) hydroxy, f) (C₁-C₄alkoxy or g)(C₁-C₄)alkylthio;
X is a) —Z or b) —B—C(R¹R²)—Z;
B is a) oxy, b) thio, c) sulfinyl, d) sulfonyl, e) methylene, or f) —N(H)—;
Z is a) —C(O)OH, b) —C(O)O-(C₁-C₄)alkyl, c) —C(O)O-(C₀-C₄)alkyl-aryl, d) —C(O)—NH₂, e) hydroxyaminocarbonyl, f) tetrazolyl, g) tetrazolylaminocarbonyl, h) 4,5-dihydro-5-oxo-1,2,4-oxadiazol-3-yl, i) 3-oxoisoxazolidin-4-yl-aminocarbonyl, j) —C(O)N(H)SO₂R⁴, or k) —NHSOR⁴; wherein R⁴ is a) (C₁-C₆)alkyl, b) amino or c) mono-N- or di-N,N-(C₁-C₆)alkylamino, wherein the (C₁-C₆)alkyl substituents in R⁴ are optionally independently substituted with one to nine fluoro;
R¹ is a) H, b) (C₁-C₄)alkyl, or c) (C₃-C₆)cycloalkyl;
R² is a) H, b) (C₃-C₆)cycloalkyl or c) a fully or partially saturated or fully unsaturated one to four membered straight or branched carbon chain; wherein the carbon(s) in the carbon chain may optionally be replaced with one or two heteroatoms selected independently from oxygen and sulfur; and wherein the sulfur is optionally mono- or di-substituted with oxo;
wherein the carbon(s) in the carbon chain in R² is optionally independently substituted as follows: a) the carbon(s) is optionally mono-, di- or tri-substituted independently with halo, b) the carbon(s) is optionally mono-substituted with hydroxy or (C₁-C₄)alkoxy, and c) the carbon(s) is optionally mono-substituted with oxo; and
wherein the carbon(s) in the carbon chain in R² is optionally mono-substituted with Q;
wherein Q is a partially or fully saturated or fully unsaturated three to eight membered ring optionally having one to four heteroatoms selected independently from oxygen, sulfur and nitrogen, or is a bicyclic ring consisting of two fused partially or fully saturated or fully unsaturated three to six membered rings, taken independently; wherein the bicyclic ring optionally has one to four heteroatoms selected independently from oxygen, sulfur and nitrogen;
wherein the Q ring is optionally mono-, di- or tri-substituted independently with a) halo, b) (C₂-C₆)alkenyl, c) (C₁-C₆) alkyl, d) hydroxy, e) (C₁-C₆)alkoxy, f) (C₁-C₄)alkylthio, g) amino, h) nitro, i) cyano, j) oxo, k) carboxy, l) (C₁-C₆)alkyloxycarbonyl, or m) mono-N- or di-N,N-(C₁-C₆)alkylamino, wherein the (C₁-C₆)alkyl and (C₁-C₆)alkoxy substituents on the Q ring is optionally mono-, di- or tri-substituted independently with a) halo, b) hydroxy, c) (C₁-C₆)alkoxy, d) (C₁-C₄)alkylthio, e) amino, f) nitro, g) cyano, h) oxo, i) carboxy, i) (C₁-C₆)alkyloxycarbonyl, or k) mono-N- or di-N,N-(C₁-C₆)alkylamino; wherein the (C₁-C₆)alkyl substituent is on the Q ring is also optionally substituted with one to nine fluoro,
or wherein R¹ and R² are linked together to form a three to six membered fully saturated carbocyclic ring, optionally having one heteroatom selected from oxygen, sulfur and nitrogen to form a heterocyclic ring;
E is a) carbonyl, b) sulfonyl, or c) methylene;
W is a) a bond, b) carbonyl, c) —N(H)—, d) —N((C₁-C₄)alkyl)-, e) (C₂-C₈)alkenyl, f) oxy, g) -(C₁-C₄alkyl-O-, h) —NH-(C₁-C₄)alkyl-, or i) -(C₁-C₆)alkyl-; wherein the (C₁-C₆)alkyl and the (C₂-C₈)alkenyl groups in W may optionally be mono- or di-substituted independently with a) oxo, b) halo, c) (C₁-C₆)alkoxycarbonyl, d) (C₁-C₆)alkyl, e) (C₂-C₆)alkenyl, f) (C₃-C₇)cycloalkyl, g) hydroxy, h) (C₁-C₆)alkoxy, i) (C₁-C₄)alkylthio, j) amino, k) cyano, l) nitro, m) mono-N- or di-N,N-(C₁-C₆)alkylamino, or n) —NH-(C₁-C)alkylamino;
or wherein W is CR⁷R⁸ wherein R⁷ and R⁸ are linked together to form a three to six membered fully saturated carbocyclic ring,
A is a) mono-N- or di-N,N-(C₁-C₆)alkylamino, b) (C₂-C₆)alkanoylamino, c) (C₁-C₆)alkoxy, d) a partially or fully saturated or fully unsaturated three to eight membered ring optionally having one to four heteroatoms selected independently from oxygen, sulfur and nitrogen, or e) a bicyclic ring consisting of two fused partially or fully saturated or fully unsaturated three to six membered rings, taken independently; wherein the bicyclic ring optionally has one to four heteroatoms selected independently from oxygen, sulfur and nitrogen; and
wherein the A ring is optionally mono-, di- or tri-substituted independently with a) oxo, b) carboxy, c) halo, d) (C₁-C₆)alkoxycarbonyl, e) (C₁-C₆)alkyl, f) (C₁-C₆)alkenyl, g) (C₃-C₇)cycloalkyl, h) (C₃-C₇)cycloalkyl(C₁-C₆)alkyl, i) hydroxy, j) (C₁-C₆)alkoxy, k) (C₁-C₄)alkylthio, l) (C₁-C₄)alkylsulfonyl, m) amino, n) cyano, o) nitro, or p) mono-N- or di-N,N-(C₁-C₆)alkylamino; wherein the (C₁-C₆)alkyl and (C₁-C₆)alkoxy substituents on the A ring are also optionally mono-, di- or tri-substituted independently with a) halo, b) hydroxy, c) (C₁-C₄)alkyl optionally substituted with one to nine fluoro, d) (C₃-C₆)cycloalkyl, e) (C₁-C₆)alkoxy, f) amino, or g) mono-N- or di-N,N-(C₁-C₆)alkylamino;
or wherein the A ring is optionally mono-substituted with a partially or fully saturated or fully unsaturated three to eight membered ring, optionally having one to four heteroatoms selected independently from oxygen, sulfur and nitrogen; also wherein this three to eight membered ring is optionally mono-, di- or tri-substituted independently with a) halo, b) hydroxy, c) (C₁-C₄)alkyl optionally substituted with one to nine fluoro, d) (C₃-C₇)cycloalkyl, e) (C₁-C₆)alkoxy optionally substituted with one to nine fluoro, f) amino, g) mono-N- or di-N,N-(C₁-C₆)alkylamino, or h) (C₁-C₄)alkylthio;
provided that:
1) when V and Y are each methylene and m and n are each one forming a six-membered piperidinyl ring, this ring is substituted by the phenyl ring (designated as J) at other than the 4-position;
2) when E is carbonyl, W is a bond and X is —B—C(R¹R²)—Z wherein R¹ and R² are each hydrogen, B is —O— or —N(H)—, and Z is —C(O)OH or —C(O)O-(C₁-C₄)alkyl, then one of F or G must be a) -(C₁-C₄)alkyl, b) (C₃-C₆)cycloalkyl, c) (C₁-C₄)alkoxy or d) (C₁-C₄)alkylthio.

18. The method according to claim 17 wherein the ruminant is a dairy cow.
